(19) United States
(12) Patent Application Publication
(10) Pub. No.: US 2022/0387414 A1
(43) Pub. Date: Dec. 8, 2022

(54) TREATING LIVER DISORDERS

(71) Applicant: Terns Pharmaceuticals Inc., Foster City, CA (US)

(72) Inventors: Martijn FENAUX, San Mateo, CA (US); Yujin WANG, San Jose, CA (US); Weidong ZHONG, San Ramon, CA (US); Kevin KLUCHER, Foster City, CA (US)

(21) Appl. No.: 17/775,242

(22) PCT Filed: Nov. 6, 2020
(86) PCT No.: PCT/US2020/059522

§ 371 Date: May 6, 2022

Related U.S. Application Data
(60) Provisional application No. 62/933,277, filed on Nov. 8, 2019.
(60) Provisional application No. 63/004,404, filed on Apr. 2, 2020.

(51) Int. Cl. A61K 31/454; A61P 1/16

(52) U.S. Cl. CPC A61K 31/454; A61P 1/16

(57) ABSTRACT

Provided herein are methods and compositions for treating liver disorders, including without limitation non-alcoholic steatohepatitis, and symptoms and manifestations thereof, in a patient.

### CROSS-REFERENCE TO RELATED APPLICATIONS

This application claims priority to U.S. Provisional Application No. 62/933,277, filed Nov. 8, 2019, and U.S. Provisional Application No. 63/004,403, filed Apr. 2, 2020, the content of each of which is incorporated herein by reference in its entirety.

### FIELD

This invention relates to methods and compositions for treating a liver disorder in a patient.

### BACKGROUND

Fatty liver disease (FLD) encompasses a spectrum of disease states characterized by excessive accumulation of fat in the liver often accompanied with inflammation. FLD can lead to non-alcoholic fatty liver disease (NAFLD), which may be characterized by insulin resistance. If untreated, NAFLD can progress to a persistent inflammatory response or non-alcoholic steatohepatitis (NASH), progressive liver fibrosis, and eventually to cirrhosis. In Europe and the US, NAFLD is the second most common reason for liver transplantation. Accordingly, the need for treatment is urgent, but due to the lack of obvious symptoms to the patient, patients may lack the motivation to maintain treatment regimens, particularly burdensome treatment regimens, such as injected medicines, medications that are administered many times a day, or any that produce dangerous or irritating side effects. There is currently no approved treatment of NASH.

### BRIEF SUMMARY

Provided herein are methods of treating a liver disorder in a patient in need thereof comprising administering a therapeutically effective amount of a compound of Formula (I)

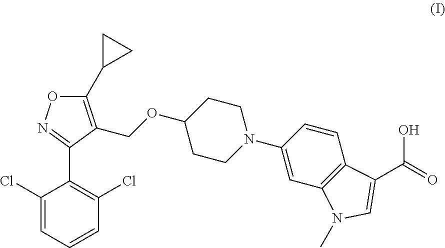 [SMILES: Cn1cc(C(=O)O)c2ccc(N3CCC(OCc4c(-c5c(Cl)cccc5Cl)noc4C4CC4)CC3)cc21]

or a pharmaceutically acceptable salt thereof, wherein the liver disorder is selected from liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH).

Also provided herein are methods of treating a liver disorder in a patient in need thereof with a Farnesoid X Receptor (FXR) agonist, comprising administering a therapeutically effective amount of the FXR agonist, wherein the FXR agonist is a compound of Formula (I)

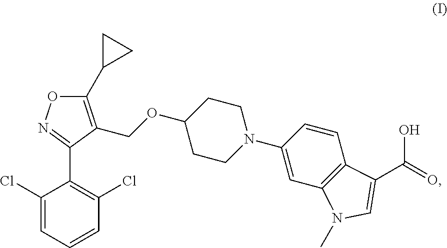 [SMILES: Cn1cc(C(=O)O)c2ccc(N3CCC(OCc4c(-c5c(Cl)cccc5Cl)noc4C4CC4)CC3)cc21]

or a pharmaceutically acceptable salt thereof, and wherein the patient has discontinued one or more prior therapies with another FXR agonist other than a compound of Formula (I), or a pharmaceutically acceptable salt thereof, wherein the liver disorder is selected from liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH). In some embodiments, the patient suffered from pruritus during the one or more prior therapies.

Also provided herein are methods of impeding or slowing the progression of non-alcoholic fatty liver disease (NAFLD) to non-alcoholic steatohepatitis (NASH) in a patient in need thereof comprising administering a therapeutically effective amount of a compound of Formula (I)

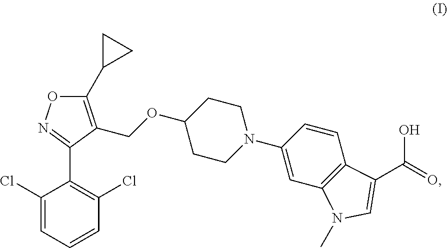 [SMILES: Cn1cc(C(=O)O)c2ccc(N3CCC(OCc4c(-c5c(Cl)cccc5Cl)noc4C4CC4)CC3)cc21]

or a pharmaceutically acceptable salt thereof.

Also provided herein are methods of impeding or slowing the progression of NASH in a patient in need thereof comprising administering a therapeutically effective amount of a compound of Formula (I)

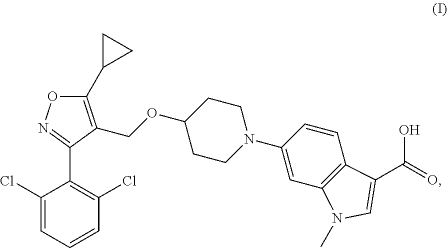 [SMILES: Cn1cc(C(=O)O)c2ccc(N3CCC(OCc4c(-c5c(Cl)cccc5Cl)noc4C4CC4)CC3)cc21]

or a pharmaceutically acceptable salt thereof.

Also provided herein are methods of treating a liver disorder in a patient in need thereof with a Farnesoid X Receptor (FXR) agonist that preferentially concentrates in liver tissue over one or more of kidney, lung, heart, and skin tissues, the method comprising administering a therapeutically effective amount of the FXR agonist, wherein the FXR agonist is a compound of Formula (I)

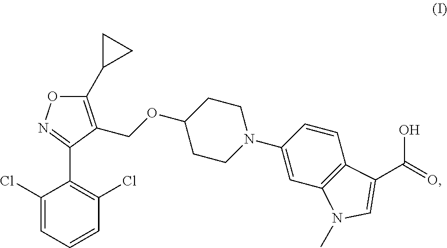 [SMILES: Cn1cc(C(=O)O)c2ccc(N3CCC(OCc4c(-c5c(Cl)cccc5Cl)noc4C4CC4)CC3)cc21]

or a pharmaceutically acceptable salt thereof, wherein the liver disorder is selected from liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH).

In some embodiments, the administration does not result in pruritus in the patient greater than Grade 2 in severity. In some embodiments, the administration does not result in pruritus in the patient greater than Grade 1 in severity. In some embodiments, the administration does not result in pruritus in the patient.

Also provided herein are methods of treating a liver disorder with an FXR agonist that does not result in detectable pruritus in a patient in need thereof, the method comprising administering a therapeutically effective amount of the FXR agonist, wherein the FXR agonist is a compound of Formula (I)

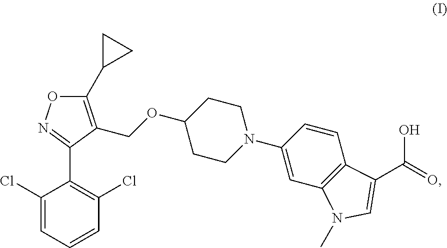 [SMILES: Cn1cc(C(=O)O)c2ccc(N3CCC(OCc4c(-c5c(Cl)cccc5Cl)noc4C4CC4)CC3)cc21]

or a pharmaceutically acceptable salt thereof, wherein the liver disorder is selected from liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH).

In some embodiments, the liver disorder is NAFLD. In some embodiments, the liver disorder is NASH. In some embodiments, the liver disorder is PSC. In some embodiments, the liver disorder is PBC.

In some embodiments, the administration results in a liver concentration to plasma concentration ratio of the compound of Formula (I) of 10 or greater.

In some embodiments, the therapeutically effective amount is 0.5 μg/day-600 mg/day. In some embodiments, the therapeutically effective amount is 0.5 μg/day-20 mg/day. In some embodiments, the therapeutically effective amount is 0.5 μg/day-4 mg/day.

In some embodiments, the administration comprises administering the compound of Formula (I), or a pharmaceutically acceptable salt thereof, daily for a treatment period of one or more weeks. In some embodiments, the compound of Formula (I), or a pharmaceutically acceptable salt thereof, is administered once daily or twice daily. In some embodiments, the amount of the compound of Formula (I), or a pharmaceutically acceptable salt thereof, administered on day 1 of the treatment period is greater than or equal to the amount administered on all subsequent days of the treatment period. In some embodiments, the amount of the compound of Formula (I), or a pharmaceutically acceptable salt thereof, administered on day 1 of the treatment period is equal to the amount administered on all subsequent days of the treatment period. In some embodiments, the treatment period is one or more months. In some embodiments, the treatment period is the remaining lifespan of the patient.

In some embodiments, the patient is obese. In some embodiments, the patient is not obese. In some embodiments, the patient also has diabetes mellitus and/or a cardiovascular disorder. In some embodiments, the patient is at risk for developing an adverse effect affecting one or more of the kidneys, lung, heart, and skin. In some embodiments, the patient is 2-17 years old. In some embodiments, the patient is 18-54 years old. In some embodiments, the patient is 65 or more years old. In some embodiments, the patient has had a liver transplant. In some embodiments, the patient's alkaline phosphatase, gamma-glutamyl transferase (GGT), alanine aminotransferase (ALT) and/or aspartate aminotransferase (AST) levels are elevated.

In some embodiments, the method does not comprise administering an antihistamine, an immunosuppressant, a steroid, rifampicin, an opioid antagonist, or a selective serotonin reuptake inhibitor (SSRI).

In some embodiments, NAS score of the patent is decreased upon administration. In some embodiments, TGR5 signaling is not activated upon administration.

In some embodiments, the administration results in decreased level of expression of a marker of fibrosis. In some embodiments, the expression level of Ccr2, Col1a1, Col1a2, Col1a3, Cxcr3, Dcn, Hgf, Il1a, Inhbe, Lox, Loxl1, Loxl2, Loxl3, Mmp2, Pdgfb, Plau, Serpine1, Perpinh1, Snai, Tgfb1, Tgfb3, Thbs1, Thbs2, Timp2, and/or Timp3 is reduced.

In some embodiments, the administration results in decreased level of expression of a marker of liver inflammation. In some embodiments, the level of Adgre1, Ccr2, Ccr5, Il1A, and/or Tlr4 is reduced.

### BRIEF DESCRIPTION OF THE DRAWINGS

FIG. 1A shows plasma concentrations of Compound I at various time points after intravenous (IV) administration to rats (1 mg/kg), dogs (1 mg/kg) and monkeys (0.3 mg/kg).

FIG. 1B shows plasma concentrations of Compound I at various time points after oral administration to mice (10 mg/kg), rats (10 mg/kg), dogs (3 mg/kg) and monkeys (5 mg/kg).

FIG. 2A shows the liver to plasma ratio of the concentration of Compound I, obeticholic acid (OCA), cilofexor, or tropifexor after 2 mg/kg IV administration to Sprague-Dawley (SD) rats.

FIG. 2B shows the tissue to plasma ratio of the concentration of Compound I for kidney, lung, and liver after 2 mg/kg IV administration of Compound I to SD rats with or without co-administration of rifampicin.

FIG. 3 shows the tissue distribution of radiolabeled Compound I in plasma, liver, small intestine, cecum, kidney, lungs, heart, and skin after 5 mg/kg oral administration of Compound I to Long-Evans rats.

FIG. 4 shows the pharmacodynamics of Compound I administration, as measured by 7-alpha-hydroxy-4-cholesten-3-one (7α-C4), after administration of 0.3 mg/kg, 1 mg/kg or 5 mg/kg oral dose to cynomolgus monkeys.

FIG. 5A shows the pharmacokinetics of Compound I administration, after administration of 1 mg/kg oral dose for one day, or 7 consecutive daily doses, to cynomolgus monkeys.

FIG. 5B shows the pharmacodynamics of Compound I administration, as measured by 7-alpha-hydroxy-4-cholesten-3-one (7α-C4), after administration of 1 mg/kg oral dose for one day, or 7 consecutive daily doses, to cynomolgus monkeys.

FIG. 6 shows RT-qPCR results measuring liver SHP1, liver OSTb, ileum SHP1, and ileum FGF15 RNA expression after administering 10 mg/kg Compound I, 30 mg/kg OCA, or vehicle control to C5BL/6 mice.

FIG. 7A shows the number of differentially expressed genes (vs. vehicle-treated: fold-change>1.5-fold; p<0.05) modulated by the administration of 10 mg/kg Compound I (500 total genes modulated) or 30 mg/kg OCA to CSBL/6 mice (44 total genes modulated), as well as the shared number differentially expressed genes that are modulated by both compounds (37 total genes).

FIG. 7B shows average expression levels (as shown by CPM value) of select FXR-related genes in CSBL/6 mice treated with 10 mg/kg Compound I or 30 mg/kg OCA, or a vehicle control.

FIG. 7C shows the number of pathways enriched (p<0.05) by the administration of 10 mg/kg Compound I (32 pathways) or 30 mg/kg OCA to CSBL/6 mice (6 pathways), as well as the number of enriched pathways by either compound (2 pathways).

FIG. 7D shows the 25 pathways most statistically enriched upon administration of 10 mg/kg Compound I to C57BL/6 mice, and compares the enrichment of those pathways to the enrichment upon administration of 30 mg/kg OCA.

FIG. 8 shows the design of a study testing the efficacy of Compound I on a mouse model of NASH.

FIG. 9 shows the NAFLD Activity Score (NAS) of control mice and mice treated with 10, 30, and 100 mg/kg Compound I.

FIG. 10A shows the steatosis score of control mice and NASH mice treated with 10, 30, and 100 mg/kg Compound I.

FIG. 10B shows the inflammation score of control mice and NASH mice treated with 10, 30, and 100 mg/kg Compound I.

FIG. 10C shows the ballooning score of control mice and NASH mice treated with 10, 30, and 100 mg/kg Compound I.

FIG. 11A shows a histological section of fibrosis in control mice and NASH mice treated with 100 mg/kg Compound I.

FIG. 11B shows the amount of fibrosis in control mice and NASH mice treated with 10, 30, and 100 mg/kg Compound I.

FIG. 12A shows the serum alanine amino transferase (ALT) levels of control mice and NASH mice treated with 10, 30, and 100 mg/kg Compound I.

FIG. 12B shows aspartate amino transferase (AST) of control mice and NASH mice treated with 10, 30, and 100 mg/kg Compound I.

FIG. 12C shows serum triglyceride levels of control mice and NASH mice treated with 10, 30, and 100 mg/kg Compound I.

FIG. 12D shows serum total cholesterol levels of control mice and NASH mice treated with 10, 30, and 100 mg/kg Compound I.

FIG. 13A shows live triglyceride levels of control mice and NASH mice treated with 10, 30, and 100 mg/kg Compound I.

FIG. 13B shows representative histology of steatosis assessment for control mice and NASH mice treated with 100 mg/kg Compound I.

FIG. 14A shows COL1A expression in the liver in control mice and NASH mice treated with 10, 30, and 100 mg/kg Compound I.

FIG. 14B shows expression levels of inflammatory genes in control mice and NASH mice treated with 30 mg/kg Compound I.

FIG. 14C shows expression of fibrosis genes in control mice and NASH mice treated with 30 mg/kg Compound I.

FIGS. 15A and 15B show mean serum concentrations of Compound I in different dosing groups on Day 1 and Day 7, respectively, after administration of Compound I.

FIGS. 16A and 16B show changes of mean serum levels of 7α-C4 from the pre-dose baseline in different dosing groups on Day 1 and Day 7, respectively, after administration of Compound I or placebo.

FIGS. 17A and 17B show changes of mean serum levels of FGF-19 from the pre-dose baseline in different dosing groups on Day 1 and Day 7, respectively, after administration of Compound I or placebo.

FIG. 18 shows changes of mean serum levels of low-density lipoprotein (LDL) from the pre-dose baseline in different dosing groups after administration of Compound I or placebo.

### DETAILED DESCRIPTION

Fatty liver disease (FLD) encompasses a spectrum of disease states characterized by excessive accumulation of fat in the liver often accompanied with inflammation. FLD can lead to non-alcoholic fatty liver disease (NAFLD), which may be characterized by insulin resistance. If untreated, NAFLD can progress to a persistent inflammatory response or non-alcoholic steatohepatitis (NASH), progressive liver fibrosis, and eventually to cirrhosis.

Fatty liver diseases, such as NAFLD and NASH, are often asymptomatic or the symptoms can be vague and difficult to define by the patient. However, in Europe and the US, NAFLD is the second most common reason for liver transplantation. Accordingly, the need for treatment is urgent, but due to the lack of obvious symptoms to the patient, patients may lack the motivation to maintain treatment regimens, particularly burdensome treatment regimens, such as injected medicines, medications that are administered many times a day, or any that produce dangerous or irritating side effects.

The Farnesoid X Receptor (FXR) is a nuclear hormone receptor that controls the conversion of cholesterol into bile acids and maintains homeostasis of multiple metabolic pathways and, therefore, is considered an important clinical target for NASH. However, FXR agonists, such as cilofexor, tropifexor, obeticholic acid (OCALIVA®), and ED-305 (Enanta) have all reported pruritus as a side effect. Pruritus can cause patient discomfort, decrease patient quality of life, and affect adherence to treatment regimens, which can be particularly of issue for conditions requiring chronic drug administration.

Provided herein are methods and compositions for treating a liver disorder in a patient in need thereof. In some embodiments, the treatment does not result in pruritus. In some embodiments, the treatment results in less pruritus than is associated with a corresponding treatment with an FXR agonist selected from the group consisting of obeticholic acid (OCA), cilofexor, or tropifexor. In some embodiments, the method comprises administering to the patient a therapeutically effective amount of a compound of Formula (I):

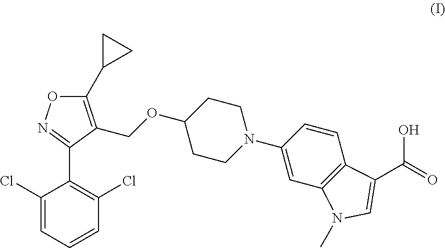 [SMILES: Cn1cc(C(=O)O)c2ccc(N3CCC(OCc4c(-c5c(Cl)cccc5Cl)noc4C4CC4)CC3)cc21]

or a pharmaceutically acceptable salt or enantiomer thereof. In some embodiments, the method comprises administering to the patient a therapeutically effective amount of a compound of Formula (I), or a pharmaceutically acceptable salt or enantiomer thereof, in combination with a therapeutically effective amount of another agent. The compound of Formula (I) is disclosed in US 2010/0152166, the content of which is incorporated by reference in its entirety, and specifically with respect to the compound of Formula (I) or a pharmaceutically acceptable salt or enantiomer thereof, as well as methods of making and using the foregoing.

In certain embodiments, the liver disorder is liver inflammation, fibrosis, or steatohepatitis. In certain embodiments, the liver disorder is selected from: liver fibrosis, alcohol induced fibrosis, steatosis, alcoholic steatosis, primary sclerosing cholangitis (PSC), primary biliary cirrhosis (PBC), NAFLD, and NASH. In some embodiments, the liver disorder is NASH. In some embodiments, the liver disorder is liver inflammation. In some embodiments, the liver disorder is liver fibrosis. In some embodiments, the liver disorder is alcohol induced fibrosis. In some embodiments, the liver disorder is steatosis. In some embodiments, the liver disorder is alcoholic steatosis. In some embodiments, the liver disorder is NAFLD. In some embodiments, the liver disorder is primary sclerosing cholangitis (PSC). In some embodiments, the liver disorder is primary biliary cirrhosis (PBC)

Also provided herein are methods of impeding or slowing the progression of NAFLD to NASH. In some embodiments, provided herein are methods of impeding or slowing the progression of NASH. NASH can progress, e.g., to one or more of liver cirrhosis, hepatic cancer, etc.

### Definitions

As used herein, the following definitions shall apply unless otherwise indicated. Further, if any term or symbol used herein is not defined as set forth below, it shall have its ordinary meaning in the art.

“Comprising” is intended to mean that the compositions and methods include the recited elements, but not excluding others. “Consisting essentially of” when used to define compositions and methods, shall mean excluding other elements of any essential significance to the combination. For example, a composition consisting essentially of the elements as defined herein would not exclude other elements that do not materially affect the basic and novel characteristic(s) of the claimed invention. “Consisting of” shall mean excluding more than trace amount of, e.g., other ingredients and substantial method steps recited. Embodiments defined by each of these transition terms are within the scope of this invention.

“Combination therapy” or “combination treatment” refers to the use of two or more drugs or agents in treatment, e.g., the use of a compound of Formula (I) together with a second agent useful to treat liver disorders, such as NAFLD, NASH, and symptoms and manifestations of each thereof is a combination therapy. Administration in “combination” refers to the administration of two agents (e.g., a compound of formula (I), and a second agent) in any manner in which the pharmacological effects of both manifest in the patient at the same time. Thus, administration in combination does not require that a single pharmaceutical composition, the same dosage form, or even the same route of administration be used for administration of both agents or that the two agents be administered at precisely the same time. Both agents can also be formulated in a single pharmaceutically acceptable composition. A non-limiting example of such a single composition is an oral composition or an oral dosage form. For example, and without limitation, it is contemplated that a compound of Formula (I) can be administered in combination therapy with a second agent in accordance with the present disclosure.

“Second agent” as used herein refers to an agent, which is other than a compound of Formula (I), and which is useful in a method described herein. The term second is meant as a term to distinguish the agent from a compound of Formula (I) or a pharmaceutically acceptable salt or enantiomer thereof, and is not intended to signify an order of administration.

The term “excipient” as used herein means an inert or inactive substance that may be used in the production of a drug or pharmaceutical, such as a tablet containing a compound of the invention as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (dc=“directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

“Patient” refers to mammals and includes humans and non-human mammals. Examples of patients include, but are not limited to mice, rats, hamsters, guinea pigs, pigs, rabbits, cats, dogs, goats, sheep, cows, and humans. In some embodiments, patient refers to a human.

“Pharmaceutically acceptable” refers to safe and non-toxic, preferably for in vivo, more preferably, for human administration.

“Pharmaceutically acceptable salt” refers to a salt that is pharmaceutically acceptable. A compound described herein may be administered as a pharmaceutically acceptable salt.

“Salt” refers to an ionic compound formed between an acid and a base. When the compound provided herein contains an acidic functionality, such salts include, without limitation, alkali metal, alkaline earth metal, and ammonium salts. As used herein, ammonium salts include, salts containing protonated nitrogen bases and alkylated nitrogen bases. Exemplary and non-limiting cations useful in pharmaceutically acceptable salts include Na, K, Rb, Cs, NH₄, Ca, Ba, imidazolium, and ammonium cations based on naturally occurring amino acids. When the compounds utilized herein contain basic functionality, such salts include, without limitation, salts of organic acids, such as carboxylic acids and sulfonic acids, and mineral acids, such as hydrogen halides, sulfuric acid, phosphoric acid, and the likes. Exemplary and non-limiting anions useful in pharmaceutically acceptable salts include oxalate, maleate, acetate, propionate, succinate, tartrate, chloride, sulfate, bisulfate, mono-, di-, and tribasic phosphate, mesylate, tosylate, and the likes.

“Therapeutically effective amount” or dose of a compound or a composition refers to that amount of the compound or the composition that results in reduction or inhibition of symptoms or a prolongation of survival in a patient. The results may require multiple doses of the compound or the composition.

“Treating” or “treatment” of a disease in a patient refers to 1) preventing the disease from occurring in a patient that is predisposed or does not yet display symptoms of the disease; 2) inhibiting the disease or arresting its development; or 3) ameliorating or causing regression of the disease.

The terms “optional” or “optionally” as used throughout the specification means that the subsequently described event or circumstance may but need not occur, and that the description includes instances where the event or circumstance occurs and instances in which it does not. For example, “the nitrogen atom is optionally oxidized to provide for the N-oxide (N→O) moiety” means that the nitrogen atom may but need not be oxidized, and the description includes situations where the nitrogen atom is not oxidized and situations where the nitrogen atom is oxidized.

### Methods of Use and Uses

Provided herein are methods of treating a liver disorder in a patient (e.g., a human patient) in need thereof comprising administering a therapeutically effective amount of a compound of Formula (I) or a pharmaceutically acceptable salt thereof, wherein the liver disorder is selected from liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH). Also provided herein are methods of treating a liver disorder in a patient (e.g., a human patient) in need thereof with an FXR agonist, comprising administering a therapeutically effective amount of the FXR agonist, wherein the FXR agonist is a compound of Formula (I) or a pharmaceutically acceptable salt thereof.

In some embodiments, the liver disorder is NAFLD or NASH. In some embodiments, the liver disorder is NAFLD. In some embodiments, the liver disorder is NASH. In some embodiments, the patient has had a liver biopsy. In some embodiments, the method further comprising obtaining the results of a liver biopsy.

Also provided herein are methods of impeding or slowing the progression of non-alcoholic fatty liver disease (NAFLD) to non-alcoholic steatohepatitis (NASH) in a patient (e.g., a human patient) in need thereof comprising administering a therapeutically effective amount of a compound of Formula (I), or a pharmaceutically acceptable salt thereof. Also provided herein are methods of impeding or slowing the progression of NASH in a patient (e.g., a human patient) in need thereof comprising administering a therapeutically effective amount of a compound of Formula (I), or a pharmaceutically acceptable salt thereof.

The compound of Formula (I), as demonstrated in the examples described herein, is preferentially distributed to the liver, which, without being bound by theory, would allow the compound to reach its FXR target in the liver with fewer off-target adverse effects. For example, the examples herein show that the compound of Formula (I) has an approximately 20-fold higher concentration in the liver than in the plasma, kidney, lungs, heart, and skin. This trait would likely be particularly beneficial for vulnerable populations, such as children, the elderly, and people with comorbidities.

Further, pruritus is a well-documented adverse effect of several FXR agonists and can result in patient discomfort, a decrease in patient quality of life, and an increased likelihood of ceasing treatment. Pruritus is particularly burdensome for indications, such as those described herein, including NASH, for which chronic drug administration is likely. The tissue specificity of the compound of Formula (I), in particular the preference for liver over skin tissue is a striking and unpredicted observation that makes it more likely that the compound will not cause pruritus in the skin, a theory that has been substantiated by human trials thus far.

Accordingly, provided herein are methods of treating a liver disorder in a patient in need thereof (e.g., a human patient) with a Farnesoid X Receptor (FXR) agonist that preferentially distributes in liver tissue over one or more of kidney, lung, heart, and skin tissues, the method comprising administering a therapeutically effective amount of the FXR agonist, wherein the FXR agonist is a compound of Formula (I), or a pharmaceutically acceptable salt thereof.

In some embodiments, provided herein are methods of treating a liver disorder in a patient in need thereof with a Farnesoid X Receptor (FXR) agonist (such as the compound of Formula (I) or a pharmaceutically acceptable salt thereof), wherein the FXR agonist does not activate TGR5 signaling. In some embodiments, the level of an FXR-regulated gene is increased. In some embodiments, the level of small heterodimer partner (SHP), bile salt export pump (BSEP) and fibroblast growth factor 19 (FGF-19) is increased. In some embodiments, the liver disorder is NASH.

In some embodiments, provided herein a method of reducing liver damage comprising administering an FXR agonist (such as a compound of Formula (I) or a pharmaceutically acceptable salt thereof) to an individual in need thereof. In some embodiments, fibrosis is reduced. In some embodiments, the level of expression of one or more markers for fibrosis is reduced. In some embodiments, the level of Ccr2, Col1a1, Col1a2, Col1a3, Cxcr3, Dcn, Hgf, Il1a, Inhbe, Lox, Loxl1, Loxl2, Loxl3, Mmp2, pdgfb, Plau, Serpine1, Perpinh1, Snai, Tgfb1, Tgfb3, Thbs1, Thbs2, Timp2, and/or Timp3 expression is reduced. In some embodiments the level of collagen is reduced. In some embodiments, the level of collagen fragments is reduced. In some embodiments, the level of expression of the fibrosis marker is reduced at least 2, at least 3, at least 4, or at least 5-fold. In some embodiments, the level of expression of the fibrosis marker is reduced about 2-fold, about 3-fold, about 4-fold, or about 5-fold.

In some embodiments, provided herein a method of reducing liver damage comprising administering an FXR agonist (such as a compound of Formula (I) or a pharmaceutically acceptable salt thereof) to an individual in need thereof. In some embodiments, inflammation is reduced. In some embodiments, one or more markers of inflammation are reduced. In some embodiments, the level of expression of Adgre1, Ccr2, Ccr5, Il1A, and/or Tlr4 is reduced. In some embodiments, the level of expression of the inflammation marker is reduced at least 2-, at least 3-, at least 4-, or at least 5-fold. In some embodiments, the level of expression of the fibrosis marker is reduced about 2-fold, about 3-fold, about 4-fold, or about 5-fold.

In some embodiments, the administration results in a liver concentration to plasma concentration ratio of the compound of Formula (I) of 10 or greater, such as 11 or greater, 12 or greater, 13 or greater, 14 or greater, or 15 or greater.

In some embodiments, the administration does not result in pruritus in the patient greater than Grade 2 in severity. In some embodiments, the administration does not result in pruritus in the patient greater than Grade 1 in severity. In some embodiments, the administration does not result in pruritus in the patient. The grading of adverse effects is known. According to Version 5 of the Common Terminology Criteria for Adverse Events (published Nov. 27, 2017), Grade 1 pruritus is characterized as “Mild or localized; topical intervention indicated.” Grade 2 pruritus is characterized as “Widespread and intermittent; skin changes from scratching (e.g., edema, papulation, excoriations, lichenification, oozing/crusts); oral intervention indicated; limiting instrumental ADL.” Grade 3 pruritus is characterized as “Widespread and constant; limiting self care ADL or sleep; systemic corticosteroid or immunosuppressive therapy indicated.” Activities of daily living (ADL) are divided into two categories: “Instrumental ADL refer to preparing meals, shopping for groceries or clothes, using the telephone, managing money, etc.,” and “Self care ADL refer to bathing, dressing and undressing, feeding self, using the toilet, taking medications, and not bedridden.”

Accordingly, also provided herein are methods of treating a liver disorder in a patient (e.g., a human patient) in need thereof with an FXR agonist that does not result in detectable pruritus in the patient in need thereof, the method comprising administering to the patient in need thereof a therapeutically effective amount of the FXR agonist, wherein the FXR agonist is a compound of Formula (I), or a pharmaceutically acceptable salt thereof.

In some embodiments, the patient is a human. Obesity is highly correlated with NAFLD and NASH, but lean people can also be affected by NAFLD and NASH. Accordingly, in some embodiments, the patient is obese. In some embodiments, the patient is not obese. Obesity can be correlated with or cause other diseases as well, such as diabetes mellitus or cardiovascular disorders. Accordingly, in some embodiments, the patient also has diabetes mellitus and/or a cardiovascular disorder. Without being bound by theory, it is believed that comorbidities, such as obesity, diabetes mellitus, and cardiovascular disorders can make NAFLD and NASH more difficult to treat. Conversely, the only currently recognized method for addressing NAFLD and NASH is weight loss, which would likely have little to no effect on a lean patient.

The risk for NAFLD and NASH increases with age, but children can also suffer from NAFLD and NASH, with literature reporting of children as young as 2 years old (Schwimmer, et al., Pediatrics, 2006, 118:1388-1393). In some embodiments, the patient is 2-17 years old, such as 2-10, 2-6, 2-4, 4-15, 4-8, 6-15, 6-10, 8-17, 8-15, 8-12, 10-17, or 13-17 years old. In some embodiments, the patient is 18-64 years old, such as 18-55, 18-40, 18-30, 18-26, 18-21, 21-64, 21-55, 21-40, 21-30, 21-26, 26-64, 26-55, 26-40, 26-30, 30-64, 30-55, 30-40, 40-64, 40-55, or 55-64 years old. In some embodiments, the patient is 65 or more years old, such as 70 or more, 80 or more, 90 or more.

NAFLD and NASH are common causes of liver transplantation, but patients that already received one liver transplant often develop NAFLD and/or NASH again. Accordingly, in some embodiments, the patient has had a liver transplant.

In some embodiments, the patient's alkaline phosphatase, gamma-glutamyl transferase (GGT), alanine aminotransferase (ALT) and/or aspartate aminotransferase (AST) levels are elevated. In some embodiments, the GGT, ALT, and/or AST levels are elevated prior to treatment with a compound of Formula (I) or a pharmaceutically acceptable salt thereof. In some embodiments, the patient's ALT level is about 2-4-fold greater than the upper limit of normal levels. In some embodiments, the patient's AST level is about 2-4-fold greater than the upper limit of normal levels. In some embodiments, the patient's GGT level is about 1.5-3-fold greater than the upper limit of normal levels. In some embodiments, the patient's alkaline phosphatase level is about 1.5-3-fold greater than the upper limit of normal levels. Methods of determining the levels of these molecules are well known. Normal levels of ALT in the blood range from about 7-56 units/liter. Normal levels of AST in the blood range from about 10-40 units/liter. Normal levels of GGT in the blood range from about 9-48 units/liter. Normal levels of alkaline phosphatase in the blood range from about 53-128 units/liter for a 20- to 50-year-old man and about 42-98 units/liter for a 20- to 50-year-old woman.

Accordingly, in some embodiments, a compound of Formula (I), or a pharmaceutically acceptable salt thereof, reduces level of AST, ALT, and/or GGT in an individual having elevated AST, ALT, and/or GGT levels. In some embodiments, the level of ALT is reduced at least 2-, at least 3-, at least 4-, or at least 5-fold. In some embodiments, the level of ALT is reduced about 2- to about 5-fold. In some embodiments, the level of AST is reduced at least 2-, at least 3-, at least 4-, or at least 5-fold. In some embodiments, the level of AST is reduced about 1.5 to about 3-fold. In some embodiments, the level of GGT is reduced at least 2, at least 3, at least 4, or at least 5-fold. In some embodiments, the level of GGT is reduced about 1.5 to about 3-fold.

In some embodiments, administration of a compound of Formula (I), or a pharmaceutically acceptable salt thereof, to a subject does not substantially change the level of low-density lipoprotein (LDL) (e.g., serum level of LDL) in the subject.

In some embodiments, administration of a compound of Formula (I), or a pharmaceutically acceptable salt thereof, to a subject results in a reduced NAFLD Activity (NAS) score. For example, in some embodiments, steatosis, inflammation, and/or ballooning is reduced upon treatment. In some embodiments, the compounds disclosed herein reduce liver fibrosis. In some embodiments, the compounds reduce serum triglycerides. In some embodiments, the compounds reduce liver triglycerides.

In some embodiments, the patient is at risk of developing an adverse effect prior to administering the compound of Formula (I) or a pharmaceutically acceptable salt thereof. In some embodiments, the adverse effect is an adverse effect which affects the kidney, lung, heart, and/or skin. In some embodiments, the adverse effect is pruritus.

In some embodiments, the patient has had one or more prior therapies. In some embodiments, the liver disorder progressed during the therapy. In some embodiments, the patient has had one or more prior therapies with another FXR agonist other that a compound of Formula (I) or a pharmaceutically acceptable salt thereof. In some embodiments, the patient suffered from pruritus during at least one of the one or more prior therapies.

In some embodiments, the therapeutically effective amount is below the level that induces an adverse effect in the patient, such as below the level that induces pruritus, such as grade 2 or grade 3 pruritus.

In some embodiments, the therapeutically effective amount of the compound of Formula (I), or a pharmaceutically acceptable salt thereof, is 500 μg/day-600 mg/day. In some embodiments, the therapeutically effective amount of the compound of Formula (I), or a pharmaceutically acceptable salt thereof, is 500 μg/day-300 mg/day. In some embodiments, the therapeutically effective amount of the compound of Formula (I), or a pharmaceutically acceptable salt thereof, is 500 μg/day-150 mg/day. In some embodiments, the therapeutically effective amount of the compound of Formula (I), or a pharmaceutically acceptable salt thereof, is 500 μg/day-100 mg/day. In some embodiments, the therapeutically effective amount of the compound of Formula (I), or a pharmaceutically acceptable salt thereof, is 500 μg/day-20 mg/day. In some embodiments, the therapeutically effective amount of the compound of Formula (I), or a pharmaceutically acceptable salt thereof, is 1 mg/day-600 mg/day. In some embodiments, the therapeutically effective amount of the compound of Formula (I), or a pharmaceutically acceptable salt thereof, is 1 mg/day-300 mg/day. In some embodiments, the therapeutically effective amount of the compound of Formula (I), or a pharmaceutically acceptable salt thereof, is 1 mg/day-150 mg/day. In some embodiments, the therapeutically effective amount of the compound of Formula (I), or a pharmaceutically acceptable salt thereof, is 1 mg/day-100 mg/day. In some embodiments, the therapeutically effective amount of the compound of Formula (I), or a pharmaceutically acceptable salt thereof, is 1 mg/day-20 mg/day. In some embodiments, the therapeutically effective amount of the compound of Formula (I), or a pharmaceutically acceptable salt thereof, is 5 mg/day-300 mg/day. In some embodiments, the therapeutically effective amount of the compound of Formula (I), or a pharmaceutically acceptable salt thereof, is 5 mg/day-150 mg/day. In some embodiments, the therapeutically effective amount of the compound of Formula (I), or a pharmaceutically acceptable salt thereof, is 5 mg/day-100 mg/day. In some embodiments, the therapeutically effective amount of the compound of Formula (I), or a pharmaceutically acceptable salt thereof, is 5 mg/day-20 mg/day. In some embodiments, the therapeutically effective amount of the compound of Formula (I), or a pharmaceutically acceptable salt thereof, is 5 mg/day-15 mg/day. In some embodiments, the therapeutically effective amount of the compound of Formula (I), or a pharmaceutically acceptable salt thereof, is 25 mg/day-300 mg/day. In some embodiments, the therapeutically effective amount of the compound of Formula (I), or a pharmaceutically acceptable salt thereof, is 25 mg/day-150 mg/day. In some embodiments, the therapeutically effective amount of the compound of Formula (I), or a pharmaceutically acceptable salt thereof, is 25 mg/day-100 mg/day. In some embodiments, the therapeutically effective amount of the compound of Formula (I), or a pharmaceutically acceptable salt thereof, is 500 μg/day-5 mg/day. In some embodiments, the therapeutically effective amount of the compound of Formula (I), or a pharmaceutically acceptable salt thereof, is 500 μg/day-4 mg/day. In some embodiments, the therapeutically effective amount of the compound of Formula (I), or a pharmaceutically acceptable salt thereof, is 5 mg/day-600 mg/day. In another embodiment, the therapeutically effective amount of the compound of Formula (I), or a pharmaceutically acceptable salt thereof, is 75 mg/day-600 mg/day.

In another embodiment, the therapeutically effective amount of the compound of Formula (I), or a pharmaceutically acceptable salt thereof, is about 1 mg/day. In another embodiment, the therapeutically effective amount of the compound of Formula (I), or a pharmaceutically acceptable salt thereof, is about 2 mg/day. In another embodiment, the therapeutically effective amount of the compound of Formula (I), or a pharmaceutically acceptable salt thereof, is about 5 mg/day. In another embodiment, the therapeutically effective amount of the compound of Formula (I), or a pharmaceutically acceptable salt thereof, is about 10 mg/day. In another embodiment, the therapeutically effective amount is about 15 mg/day. In another embodiment, the therapeutically effective amount of the compound of Formula (I), or a pharmaceutically acceptable salt thereof, is about 25 mg/day. In another embodiment, the therapeutically effective amount of the compound of Formula (I), or a pharmaceutically acceptable salt thereof, is about 75 mg/day. In another embodiment, the therapeutically effective amount of the compound of Formula (I), or a pharmaceutically acceptable salt thereof, is about 200 mg/day. In another embodiment, the therapeutically effective amount of the compound of Formula (I), or a pharmaceutically acceptable salt thereof, is about 400 mg/day. In another embodiment, the therapeutically effective amount of the compound of Formula (I), or a pharmaceutically acceptable salt thereof, is about 600 mg/day.

In some embodiments, the administration comprises administering the compound of Formula (I), or a pharmaceutically acceptable salt thereof, daily for a treatment period of one or more weeks. In some embodiments, the administration comprises administering the compound of Formula (I), or a pharmaceutically acceptable salt thereof, twice daily for a treatment period of one or more weeks. In some embodiments, the administration comprises administering the compound of Formula (I), or a pharmaceutically acceptable salt thereof, every other day for a treatment period of one or more weeks.

In some embodiments, the amount of the compound of Formula (I), or a pharmaceutically acceptable salt thereof, administered on day 1 of the treatment period is greater than or equal to the amount administered on all subsequent days of the treatment period. In some embodiments, the amount of the compound of Formula (I), or a pharmaceutically acceptable salt thereof, administered on day 1 of the treatment period is equal to the amount administered on all subsequent days of the treatment period.

In some embodiments, the treatment period is at least 1 week, 2 weeks, 3 weeks, 4 weeks, 5 weeks, 1 month, 2 months, 3 months, 4 months, 5 months, 6 months, 7 months, 8 months, 9 months, 10 months, 11 months, 12 months, 1 year, 2 years, 3 years, 4 years, or more. In some embodiments, the treatment period is from about a week to about a month, from about a month to about a year, from about a year to about several years. In some embodiments, the treatment period at least any of about 1 week, 2 weeks, 3 weeks, 4 weeks, 5 weeks, 1 month, 2 months, 3 months, 4 months, 5 months, 6 months, 7 months, 8 months, 9 months, 10 months, 11 months, 12 months, 1 year, 2 years, 3 years, 4 years, or more. In some embodiments, the treatment period is the remaining lifespan of the patient.

In some embodiments, the compound of Formula (I), or a pharmaceutically acceptable salt thereof, is administered with rifampicin. In some embodiments, the compound of Formula (I), or a pharmaceutically acceptable salt thereof, is administered as a monotherapy, i.e., administered in absence of another agent, which: is useful in treating or substantially treating a liver disorder, impedes or slows the progression of non-alcoholic fatty liver disease (NAFLD) to non-alcoholic steatohepatitis (NASH); or impedes or slows the progression of NASH, in a patient in need thereof. In some embodiments, the method does not comprise treating pruritus in the patient. In some embodiments, the method does not comprise administering an antihistamine, an immunosuppressant, a steroid (such as a corticosteroid), rifampicin, an opioid antagonist, or a selective serotonin reuptake inhibitor (SSRI).

In some embodiments, the therapeutically effective amount of the compound of Formula (I), or a pharmaceutically acceptable salt thereof, is administered once daily. In some embodiments, the therapeutically effective amount of the compound of Formula (I), or a pharmaceutically acceptable salt thereof, is administered twice daily. In some embodiments, the therapeutically effective amount of the compound of Formula (I), or a pharmaceutically acceptable salt thereof, is 75 mg-200 mg twice daily per patient. In some embodiments, the compound of Formula (I), or a pharmaceutically acceptable salt thereof, is administered as a pharmaceutically acceptable composition comprising at least one pharmaceutically acceptable excipient, carrier, or diluent.

In some embodiments, the administration modulates one or more of the following: a metabolic pathway, bile secretion, retinol metabolism, drug metabolism-cytochrome P450, fat digestion and absorption, glycerolipid metabolism, chemical carcinogenesis, glyceropholipid metabolism, nicotine addiction, linoleic acid metabolism, ABC transporters, metabolism of xenobiotics by cytochrome P450, sphingolipid metabolism, glutathione metabolism, folate biosynthesis, morphine addiction, glycosphingolipid biosynthesis-lacto and neolacto series, arachidonic acid metabolism, tyrosine metabolism, maturity onset diabetes of the young, DNA replication, cholesterol metabolism, drug metabolism-other enzymes, and ether lipid metabolism. In some embodiments,-the administration modulates one or more of the following: a metabolic pathway, retinol metabolism, fat digestion and absorption, glycerolipid metabolism, chemical carcinogenesis, glyceropholipid metabolism, ABC transporters, metabolism of xenobiotics by cytochrome P450, sphingolipid metabolism, glutathione metabolism, folate biosynthesis, and morphine addiction. In some embodiments, the administration modulates expression of one or more of the following: Abcb4, Apoa5, Cyp7a1, Cyp8b1, Nr0b2, and Sic51b.

### Compounds

In some embodiments, the compound utilized herein is of Formula (I):

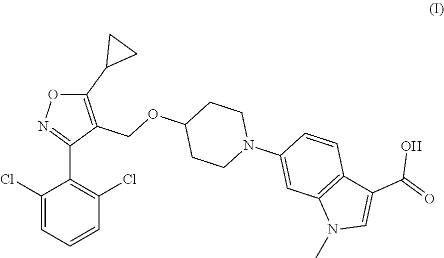 [SMILES: Cn1cc(C(=O)O)c2ccc(N3CCC(OCc4c(-c5c(Cl)cccc5Cl)noc4C4CC4)CC3)cc21]

or a tautomer thereof, or an isotopomer of each thereof, or an enantiomer or diastereomer of the foregoing, or a pharmaceutically acceptable salt of each of the above. In some embodiments, the compound utilized herein is the compound of Formula (I). In some embodiments, the compound utilized herein is a pharmaceutically acceptable salt of the compound of Formula (I).

The compounds utilized herein may be prepared by a combination of a variety of stepwise procedures known in the art, such as, e.g., US 2010/0152166 (incorporated herein by reference).

In some embodiments, the compound of Formula (I) is 6-{4-[5-cyclopropyl-3-(2,6-dichloro-phenyl)-isoxazol-4-ylmethoxy]-piperidin-1-yl}-1-methyl-1H-indole-3-carboxylic acid:

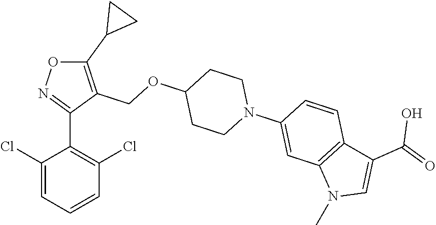 [SMILES: Cn1cc(C(=O)O)c2ccc(N3CCC(OCc4c(-c5c(Cl)cccc5Cl)noc4C4CC4)CC3)cc21]

or a pharmaceutically acceptable salt or enantiomer thereof.

### Compositions

A compound as detailed herein may in some aspects be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form. In one variation, “substantially pure” intends a composition that contains no more than 35% impurity, wherein the impurity denotes a compound other than the compound comprising the majority of the composition or a salt thereof. For example, a composition of a substantially pure compound intends a composition that contains no more than 35% impurity, wherein the impurity denotes a compound other than the compound or a salt thereof. In one variation, a composition of substantially pure compound or a salt thereof is provided wherein the composition contains no more than 25% impurity. In another variation, a composition of substantially pure compound or a salt thereof is provided wherein the composition contains no more than 20% impurity. In still another variation, a composition of substantially pure compound or a salt thereof is provided wherein the composition contains no more than 10% impurity. In a further variation, a composition of substantially pure compound or a salt thereof is provided wherein the composition contains no more than 5% impurity. In another variation, a composition of substantially pure compound or a salt thereof is provided wherein the composition contains no more than 3% impurity. In still another variation, a composition of substantially pure compound or a salt thereof is provided wherein the composition contains no more than 1% impurity. In a further variation, a composition of substantially pure compound or a salt thereof is provided wherein the composition contains no more than 0.5% impurity. In yet other variations, a composition of substantially pure compound means that the composition contains no more than 15%, such as no more than 10%, no more than 5%, no more than 3%, or no more than 1% impurity, which impurity may be the compound in a different stereochemical form. For instance, and without limitation, a composition of substantially pure (S) compound means that the composition contains no more than 15% or no more than 10% or no more than 5% or no more than 3% or no more than 1% of the (R) form of the compound.

In one variation, the compounds herein are synthetic compounds prepared for administration to an individual such as a human. In another variation, compositions are provided containing a compound in substantially pure form. In another variation, the disclosure embraces pharmaceutical compositions comprising a compound detailed herein and a pharmaceutically acceptable carrier or excipient. In another variation, provided herein are compositions or pharmaceutical compositions for use in any of the methods of provided herein. The purified forms, pharmaceutical compositions and methods of administering the compounds are suitable for any compound or form thereof detailed herein.

Pharmaceutical compositions of any of the compounds detailed herein are embraced by this disclosure. Thus, the disclosure includes pharmaceutical compositions comprising a compound of Formula (I), or a pharmaceutically acceptable salt thereof, and a pharmaceutically acceptable carrier or excipient. In one aspect, the pharmaceutically acceptable salt is an acid addition salt, such as a salt formed with an inorganic or organic acid. Pharmaceutical compositions according to the invention may take a form suitable for oral, buccal, parenteral, nasal, topical or rectal administration or a form suitable for administration by inhalation.

The compound or pharmaceutical composition may be formulated for any available delivery route, including an oral, mucosal (e.g., nasal, sublingual, vaginal, buccal or rectal), parenteral (e.g., intramuscular, subcutaneous or intravenous), topical or transdermal delivery form. A compound or pharmaceutical composition may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

The compound described herein can be used in the preparation of a composition, such as a pharmaceutical composition, by combining the compound as an active ingredient with a pharmaceutically acceptable carrier, such as those mentioned above. Depending on the therapeutic form of the system (e.g., transdermal patch vs. oral tablet), the carrier may be in various forms. In addition, pharmaceutical compositions may contain preservatives, solubilizers, stabilizers, re-wetting agents, emulgators, sweeteners, dyes, adjusters, and salts for the adjustment of osmotic pressure, buffers, coating agents or antioxidants.

Compositions comprising the compound may also contain other substances which have valuable therapeutic properties. Pharmaceutical compositions may be prepared by known pharmaceutical methods. Suitable formulations can be found, e.g., in Remington: The Science and Practice of Pharmacy, Lippincott Williams & Wilkins, 21^{st} ed. (2005), which is incorporated herein by reference.

Compounds as described herein may be administered to individuals (e.g., a human) in a form of generally accepted oral compositions, such as tablets, coated tablets, and gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid polyols, and so on. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants.

Any of the compounds described herein can be formulated in a tablet in any dosage form described.

Compositions comprising a compound provided herein are also described. In one variation, the composition comprises a compound and a pharmaceutically acceptable carrier or excipient. In another variation, a composition of substantially pure compound is provided.

In some embodiments, provided herein is a pharmaceutically acceptable composition comprising a compound of Formula (I), or a tautomer thereof, or an isotopomer of each thereof, or an enantiomer or diastereomer of the foregoing, or a pharmaceutically acceptable salt of each of the above, and at least one pharmaceutically acceptable excipient, carrier, or diluent for treating a liver disorder; for impeding or slowing the progression of non-alcoholic fatty liver disease (NAFLD) to non-alcoholic steatohepatitis (NASH); or for impeding or slowing the progression of NASH, in a patient in need thereof, wherein the liver disorder is selected from liver inflammation, liver fibrosis, alcohol induced fibrosis, steatosis, alcoholic steatosis, primary sclerosing cholangitis (PSC), primary biliary cirrhosis (PBC), NAFLD, and NASH.

In some embodiments, provided herein is a unit dose form of the pharmaceutically acceptable formulations provided herein. In some embodiments, the unit dose form comprises a therapeutically effective amount of a compound of Formula (I). In some embodiments, the unit dose form comprises a therapeutically effective amount of a compound of Formula (I) and a therapeutically effective amount of another agent. In some embodiments, the unit dose form is for treating a liver disorder; for impeding or slowing the progression of non-alcoholic fatty liver disease (NAFLD) to non-alcoholic steatohepatitis (NASH); or for impeding or slowing the progression of NASH, in a patient in need thereof, wherein the liver disorder is selected from liver inflammation, liver fibrosis, alcohol induced fibrosis, steatosis, alcoholic steatosis, primary sclerosing cholangitis (PSC), primary biliary cirrhosis (PBC), NAFLD, and NASH.

Also provided herein are uses of a compound of Formula (I), or a pharmaceutically acceptable salt thereof, in the manufacture of a medicament. In some embodiments, the manufacture of a medicament is for the treatment of a liver disorder selected from the group consisting of liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH). In some embodiments, the manufacture of a medicament is for the treatment of non-alcoholic steatohepatitis (NASH).

### Exemplary Embodiments

Among the Provided Embodiments are:

1. A method of treating a liver disorder in a patient in need thereof comprising administering a therapeutically effective amount of a compound of Formula (I)

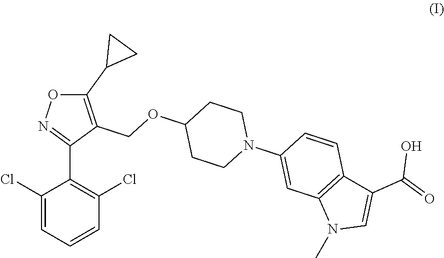 [SMILES: Cn1cc(C(=O)O)c2ccc(N3CCC(OCc4c(-c5c(Cl)cccc5Cl)noc4C4CC4)CC3)cc21]

or a pharmaceutically acceptable salt thereof, wherein the liver disorder is selected from liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH).

2. A method of treating a liver disorder in a patient in need thereof with a Farnesoid X Receptor (FXR) agonist, comprising administering a therapeutically effective amount of the FXR agonist, wherein the FXR agonist is a compound of Formula (I)

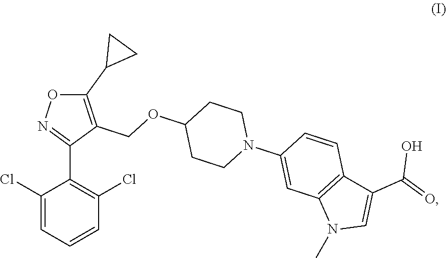 [SMILES: Cn1cc(C(=O)O)c2ccc(N3CCC(OCc4c(-c5c(Cl)cccc5Cl)noc4C4CC4)CC3)cc21]

or a pharmaceutically acceptable salt thereof, and wherein the patient has discontinued one or more prior therapies with another FXR agonist other than a compound of Formula (I) or a pharmaceutically acceptable salt thereof, wherein the liver disorder is selected from liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH).

3. The method of embodiments 2, wherein the patient suffered from pruritus during the one or more prior therapies.

4. A method of impeding or slowing the progression of non-alcoholic fatty liver disease (NAFLD) to non-alcoholic steatohepatitis (NASH) in a patient in need thereof comprising administering a therapeutically effective amount of a compound of Formula (I)

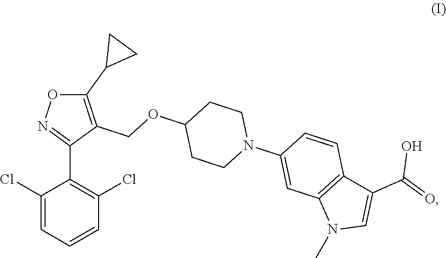 [SMILES: Cn1cc(C(=O)O)c2ccc(N3CCC(OCc4c(-c5c(Cl)cccc5Cl)noc4C4CC4)CC3)cc21]

or a pharmaceutically acceptable salt thereof.

5. A method of impeding or slowing the progression of NASH in a patient in need thereof comprising administering a therapeutically effective amount of a compound of Formula (I)

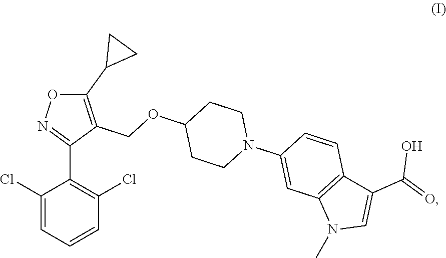 [SMILES: Cn1cc(C(=O)O)c2ccc(N3CCC(OCc4c(-c5c(Cl)cccc5Cl)noc4C4CC4)CC3)cc21]

or a pharmaceutically acceptable salt thereof.

6. A method of treating a liver disorder in a patient in need thereof with a Farnesoid X Receptor (FXR) agonist that preferentially concentrates in liver tissue over one or more of kidney, lung, heart, and skin tissues, the method comprising administering a therapeutically effective amount of the FXR agonist, wherein the FXR agonist is a compound of Formula (I)

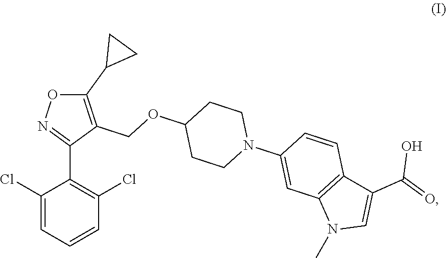 [SMILES: Cn1cc(C(=O)O)c2ccc(N3CCC(OCc4c(-c5c(Cl)cccc5Cl)noc4C4CC4)CC3)cc21]

or a pharmaceutically acceptable salt thereof, wherein the liver disorder is selected from liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH).

7. The method of any one of embodiments 1-6, wherein the administration does not result in pruritus in the patient greater than Grade 2 in severity.

8. The method of any one of embodiments 1-7, wherein the administration does not result in pruritus in the patient greater than Grade 1 in severity.

9. The method of any one of embodiments 1-8, wherein the administration does not result in pruritus in the patient.

10. A method of treating a liver disorder with an FXR agonist that does not result in detectable pruritus in a patient in need thereof, the method comprising administering a therapeutically effective amount of the FXR agonist, wherein the FXR agonist is a compound of Formula (I)

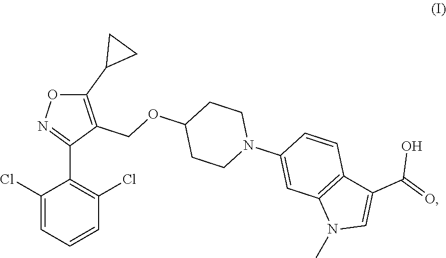 [SMILES: Cn1cc(C(=O)O)c2ccc(N3CCC(OCc4c(-c5c(Cl)cccc5Cl)noc4C4CC4)CC3)cc21]

or a pharmaceutically acceptable salt thereof, wherein the liver disorder is selected from liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH).

11. The method of any one of embodiments 1-3, or 6-10, wherein the liver disorder is NAFLD.

12. The method of any one of embodiments 1-3, or 6-10, wherein the liver disorder is NASH.

13. The method of any one of embodiments 1-3, or 6-10, wherein the liver disorder is PSC.

14. The method of any one of embodiments 1-3, or 6-10, wherein the liver disorder is PBC.

15. The method of any one of embodiments 1-14, wherein the administration results in a liver concentration to plasma concentration ratio of the compound of Formula (I) of 10 or greater.

16. The method of any one of embodiments 1-15, wherein the therapeutically effective amount is 0.5 μg/day-600 mg/day.

17. The method of any one of embodiments 1-16, wherein the therapeutically effective amount is 0.5 μg/day-20 mg/day.

18. The method of any one of embodiments 1-17, wherein the therapeutically effective amount is 0.5 μg/day-4 mg/day.

19. The method of any one of embodiments 1-18, wherein the administration comprises administering the compound of Formula (I), or a pharmaceutically acceptable salt thereof, daily for a treatment period of one or more weeks.

20. The method of embodiment 19, wherein the compound of Formula (I), or a pharmaceutically acceptable salt thereof, is administered once daily or twice daily.

21. The method of embodiment 19 or 20, wherein the amount of the compound of Formula (I), or a pharmaceutically acceptable salt thereof, administered on day 1 of the treatment period is greater than or equal to the amount administered on all subsequent days of the treatment period.

22. The method of any one of embodiments 19-21, wherein the amount of the compound of Formula (I), or a pharmaceutically acceptable salt thereof, administered on day 1 of the treatment period is equal to the amount administered on all subsequent days of the treatment period.

23. The method of any one of embodiments 1-22, wherein the treatment period is one or more months.

24. The method of any one of embodiments 1-23, wherein the treatment period is the remaining lifespan of the patient.

25. The method of any one of embodiments 1-24, wherein the patient is obese.

26. The method of any one of embodiments 1-24, wherein the patient is not obese.

27. The method of any one of embodiments 1-26, wherein the patient also has diabetes mellitus and/or a cardiovascular disorder.

28. The method of any one of embodiments 1-27, wherein the patient is at risk for developing an adverse effect affecting one or more of the kidneys, lung, heart, and skin.

29. The method of any one of embodiments 1-28, wherein the patient is 2-17 years old.

30. The method of any one of embodiments 1-28, wherein the patient is 18-54 years old.

31. The method of any one of embodiments 1-28, wherein the patient is 65 or more years old.

32. The method of any one of embodiments 1-31, wherein the patient has had a liver transplant.

33. The method of any one of embodiments 1-32, wherein the patient's alkaline phosphatase, gamma-glutamyl transferase (GGT), alanine aminotransferase (ALT) and/or aspartate aminotransferase (AST) levels are elevated.

34. The method of any one of embodiments 1-33, wherein the method does not comprise administering an antihistamine, an immunosuppressant, a steroid, rifampicin, an opioid antagonists, or a selective serotonin reuptake inhibitor (SSRI).

### EXAMPLES

The application may be better understood by reference to the following non-limiting examples, which are provided as exemplary embodiments of the application. The following examples are presented in order to more fully illustrate embodiments and should in no way be construed as limiting the scope of the application. While certain embodiments of the present application have been shown and described herein, it will be obvious that such embodiments are provided by way of example only. Numerous variations, changes, and substitutions may occur to those skilled in the art without departing from the spirit and scope of the invention. It should be understood that various alternatives to the embodiments described herein may be employed in practicing the methods described herein.

As used herein, “Compound I” refers to the compound of Formula (I)

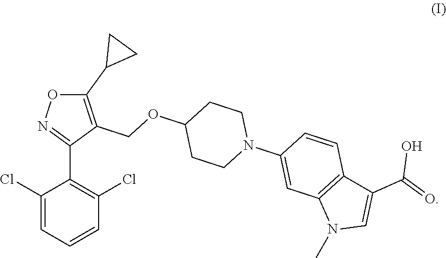 [SMILES: Cn1cc(C(=O)O)c2ccc(N3CCC(OCc4c(-c5c(Cl)cccc5Cl)noc4C4CC4)CC3)cc21]

### Example 1: In Vitro Metabolic Stability

The rate of hepatic metabolism of Compound I was assessed in cryopreserved hepatocytes to determine the in vitro half-life of the compound. 1 μM of Compound I was mixed with preconditioned mouse, rat, dog, monkey, or human hepatocytes (0.5×10⁶ cells/mL) and allowed to incubate at 37° C. for 2 hours, with samples collected at several time points and assayed for Compound I. In vitro half-life values were determined and scaled to predict hepatic clearance (CL_{pred}) and hepatic extraction using the well-stirred liver model with no correction for plasma protein as described in Obach et al., The Prediction of Human Pharmacokinetic Parameters from Preclinical and In Vitro Metabolism Data, J. of Pharmacology and Experimental Therapeutics, vol. 283, no. 1, pp. 46-58 (1997). Results are shown in Table 1, which demonstrate that Compound I was moderately metabolized in hepatocytes of all tested species.

TABLE 1
In Vitro metabolic stability of Compound I

| | | | Hepatic |
|---|---|---|---|
| | t_{1/2} | In vitro Metabolic | Extraction |
| Species | (min) | CL_{pred} (L/h/kg) | (%) |
| Mouse | 43.6 ± 2.83 | 4.36 ± 0.06 | 80.7 ± 1.02 |
| Sprague- | 131 ± 4.11 | 1.57 ± 0.03 | 47.3 ± 0.78 |
| Dawley Rat | | | |
| Beagle Dog | 126 ± 15.5 | 1.32 ± 0.05 | 71.0 ± 2.49 |
| Cynomolgus | 63.4 ± 0.78 | 1.68 ± 0.01 | 64.4 ± 0.28 |
| Monkey | | | |
| Human | 84.1 ± 6.48 | 0.83 ± 0.22 | 67.0 ± 1.73 |

### Example 2: In Vitro OATP Transport Assay

A polarized monolayer of MDCK-II cells grown on a permeable support was used to test the ability of organic-anion-transporting polypeptide (OATP) 1B1 or OATP 1B3 to transport Compound I across the lipid bilayer and into the cells. The MDCK-II cells were transfected one of (1) a vector to express OATP 1B1, (2) a vector to express OATP 1B3, or (3) a control vector. Expression was induced in the cells before culturing the cells at 37° C. in 5% CO₂ atmosphere. After inducing expression, the cells were treated with 1 μM, 3 μM, and 10 Compound I, or 3 μM Compound I and 100 μM rifampin. Cellular uptake of Compound I was then measured. Results from this experiment demonstrated that Compound I is not an OATP 1B1 or OATP 1B3 substrate.

### Example 3: Pharmacokinetics Assay

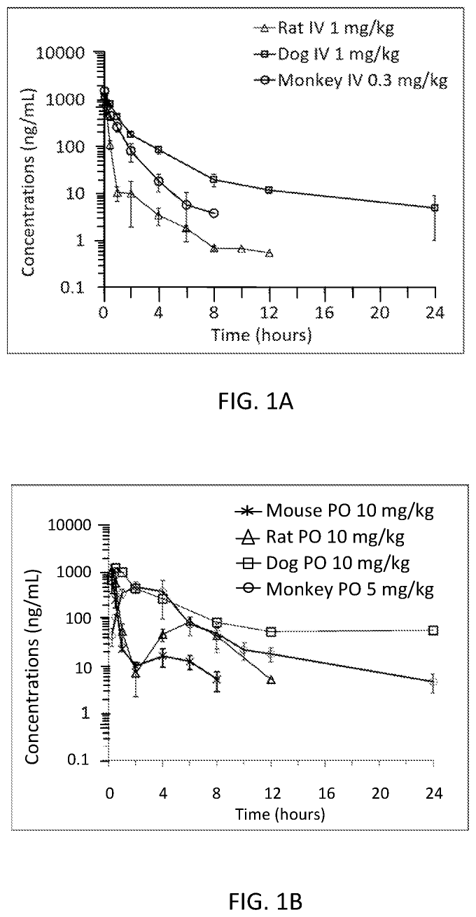

Compound I was administered to Sprague-Dawley (SD) rats intravenously at 1 mg/kg (n=3) or orally at 10 mg/kg (n=3), to beagle dogs intravenously at 1 mg/kg (n=3) or orally at 3 mg/kg (n=3), to cynomolgus monkeys intravenously at 0.3 mg/kg (n=6) or orally at 5 mg/kg (n=6), and to mice orally at 5 mg/kg (n=9). Compound I for oral administration to SD rats was formulated in a vehicle containing 10% DMSO, 10% Cremophor-EL, and 80% aqueous solution (10% 2-hydroxypropyl-β-cyclodextrin). Compound I for oral administration to beagle dogs was formulated with an aqueous solution containing 1% carboxymethyl cellulose, 0.25% Tween-80, and 0.05% antifoam. Compound I for oral administration to cynomolgus monkeys was formulated with 10% Solutol, 20% PEG400, 0.5% Tween-80 and 69.5% deionized water. Serial blood samples were collected, and plasma concentrations of the Compound I were measured. Results are shown in FIG. 1A (IV administration) and FIG. 1B (oral administration), and in Table 2. The results demonstrate that Compound I has low to moderate clearance in vivo. The volume of distribution (V_{dss}) of Compound I is greater than the volume of total body water (0.70 L/kg) in rat and dog. Smaller V_{dss} in monkeys is correlated with higher plasma protein binding.

TABLE 2
Pharmacokinetic parameters of Compound I

| | | | IV Terminal | Oral |
|---|---|---|---|---|
| | CL | V_{dss} | t_{1/2} | Bioavailability |
| Species | (L/h/kg) | (L/kg) | (h) | (%) |
| Sprague- | 2.55 | 1.31 | 2.45 | 21 |
| Dawley Rat | | | | |
| Beagle Dog | 0.54 | 1.92 | 5.67 | 82 |
| Cynomolgus | 0.30 | 0.6 | 1.32 | 18 |
| Monkey | | | | |

### Example 5: Tissue Distribution of Compound I

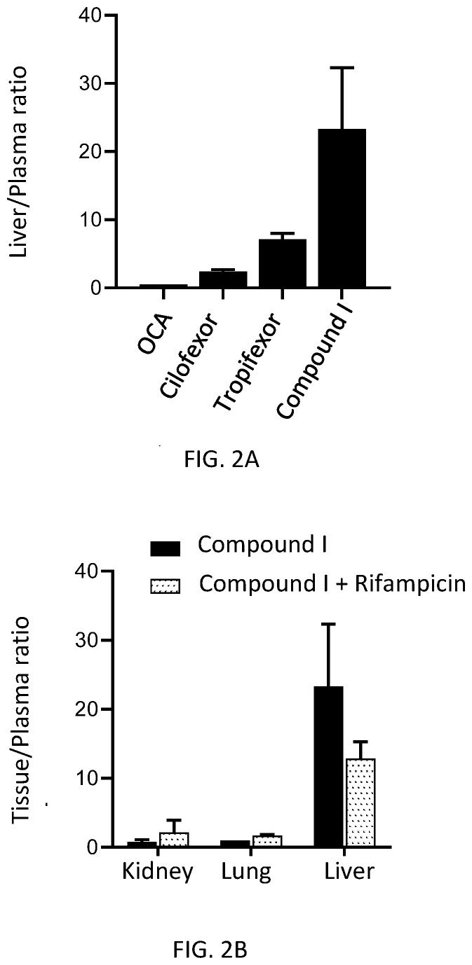

Tissue distribution of Compound I administered to rats was determined and compared to distribution other Farnesoid X Receptor (FXR) agonists cilofexor, tropifexor, and obeticholic acid (OCA). The tested compounds were administered to SD rats (n=3 per compound) by way of 30 minute intravenous infusion at 2 mg/kg. Blood, liver, kidney, and lung tissue samples were collected from the rats to determine a tissue/plasma ratio. The liver tissue/plasma ratio for the compounds is shown in FIG. 2A, which demonstrates that substantially more of Compound I localizes to the liver tissue compared to the other tested compounds. Co-administration of Compound I with 100 μM rifampin does not result in a significant change in distribution of Compound I to the liver (FIG. 2B). These results collectively demonstrated that Compound I is preferentially distributed to the liver and exhibited high liver/plasma ratio in rodent species, approximately 3 to 20-fld higher than other FXR agonists being studied for the treatment of NASH (cilofexor, tropifexor, and OCA).

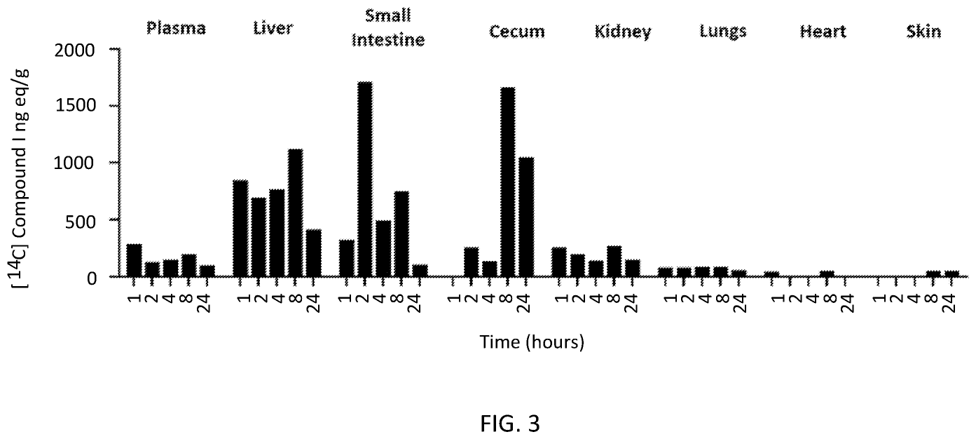

Radiolabeled (¹⁴C) Compound I was also administered to Long-Evans rats at an oral dose of 5 mg/kg (100 μCi/kg). Plasma, liver, small intestine, cecum, kidney, lung, heart and skin tissue samples were collected up to 168 hours, and the amount of radioactive material at various time points was measured. Results are shown in FIG. 3. Liver, small intestine, and cecum had the most radioactive material.

### Example 6: Metabolism of Compound I

Radiolabeled (¹⁴C) Compound I was administered to bile duct intact or cannulated SD rats orally at 5 mg/kg or intravenously at 2 mg/kg (n=3 for each of the four cohorts) for a total radioactive dose of 100 μCi/kg. Blood, bile, feces, and urine samples were collected from each rat for up to 168 hours. Compound I was metabolized into an acyl glucuronide metabolite prior to biliary excretion, which was determined as the major elimination pathway for the compound.

### Example 7: Pharmacokinetics/Pharmacodynamics Profile

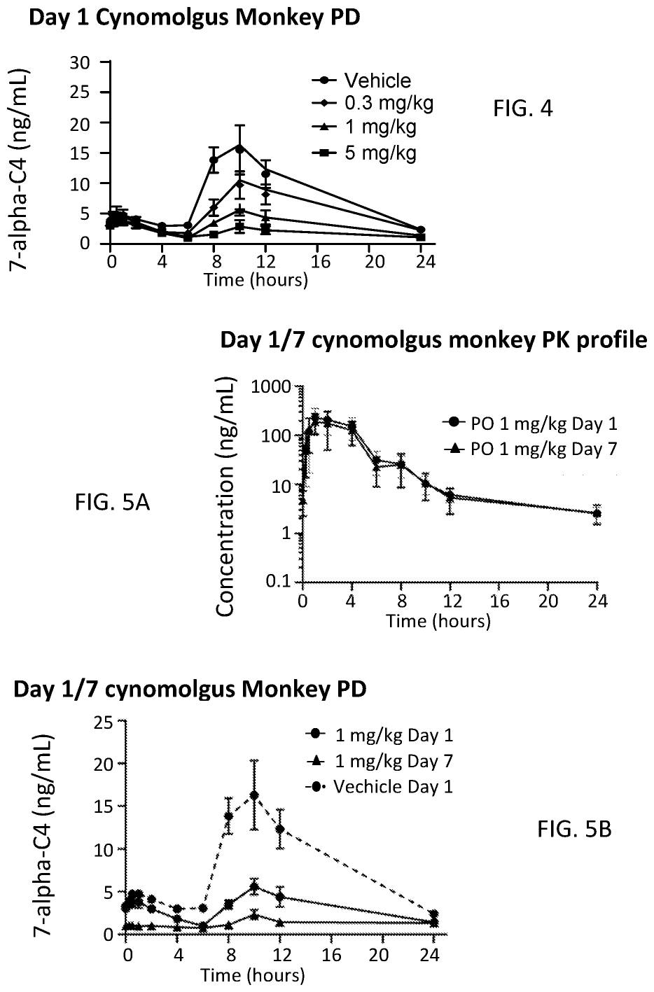

Pharmacokinetics/pharmacodynamics (PK/PD) profiles for cynomolgus monkeys was determined by administering an oral dose of Compound I suspension at doses of 0 (vehicle), 0.3, 1, or 5 mg/kg, and collecting blood samples for up to 24 hours. The pharmacodynamics were measured as a function of 7-alpha-hydroxy-4-cholesten-3-one (7α-C4) reduction (FIG. 4), as quantified by LC-MS/MS. Pharmacokinetics data is presented in Table 3, and were determined by non-compartmental analysis.

TABLE 3
Pharmacokinetic parameters of Compound I

| | PK Parameters | | |
|---|---|---|---|
| Compound I | AUC₀₋₂₄ | Cₘₐₓ | Tₘₐₓ |
| dose | (ng*hr/mL) | (ng/mL) | (hr) |
| 0.3 mg/kg | 196 ± 64 | 58.8 ± 30.2 | 2.17 ± 1.47 |
| 1 mg/kg | 1000 ± 419 | 257 ± 124 | 1.83 ± 1.17 |
| 5 mg/kg | 2720 ± 1500 | 709 ± 458 | 2.25 ± 1.47 |

Compound I was also orally administered at 1 mg/kg for 7 consecutive days to cynomolgus monkeys (n=6) to determine the PK/PD profile following multiple doses. Results of this study are shown in FIG. 5A (PK profile) and FIG. 5B (PD profile) and Table 4, and demonstrate that the plasma exposure of Compound I was comparable on day 1 and day 7 and that sustained suppression of the pharmacodynamics biomarker 7α-C4 was achieved after repeated oral dosing.

TABLE 4
Pharmacokinetic parameters of Compound I

| PK | Cₘₐₓ | AUC₀₋₂₄ | Tₘₐₓ |
|---|---|---|---|
| Parameters | (ng/mL) | (ng*hr/mL) | (hr) |
| Day 1 | 257 ± 124 | 1000 ± 419 | 1.83 ± 1.17 |
| Day 7 | 221 ± 121 | 858 ± 425 | 1.25 ± 0.61 |

### Example 8: Mechanism of Action

C5BL/6 mice were administered a single oral dose of 10 mg/kg Compound I (n=6), 30 mg/kg OCA (n=6), or a vehicle control (n=6), and tissue RNA samples were collected 6 hours after dose administration. The RNA was analyzed by RT-qPCR and RNAseq.

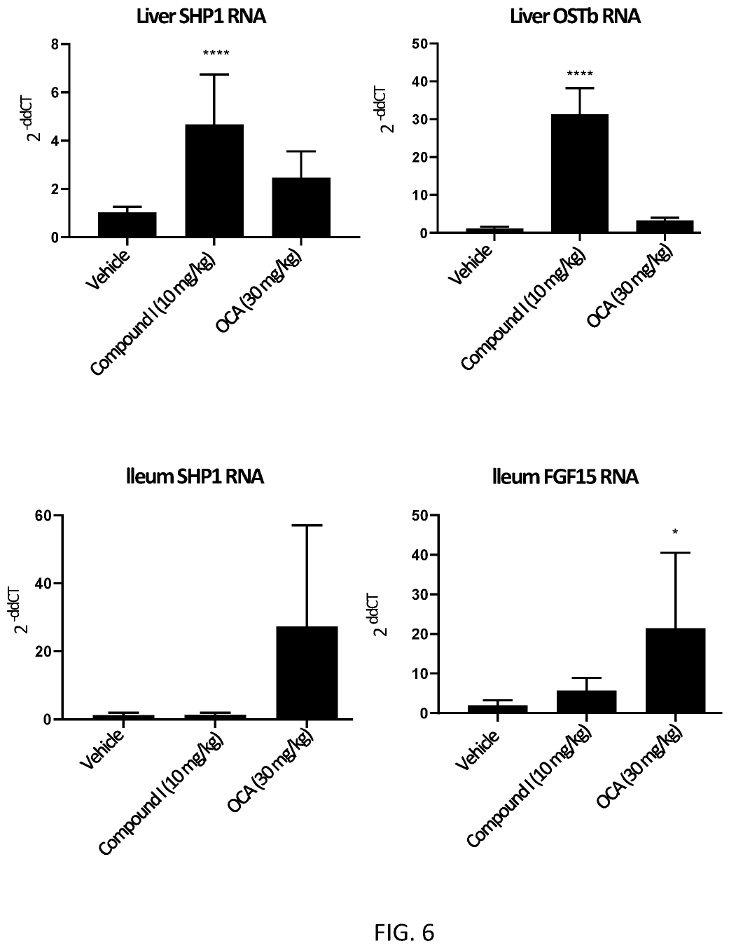

For RT-qPCR, gene-specific primers were used to quantitate FXR-regulated gene expression in liver and ileum using the 2-ddCT method. Results are shown in FIG. 6 (data presented as mean±SEM; **** indicates p<0.0001 and * indicates p<0.05 versus vehicle, with statistics determined by one-way ANOVA followed by Tukey). This data indicates that Compound I preferentially induces FXR-specific genes in the liver of mice.

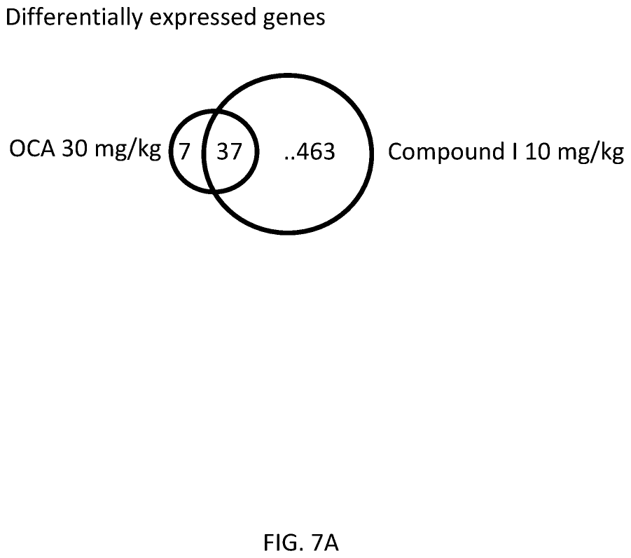

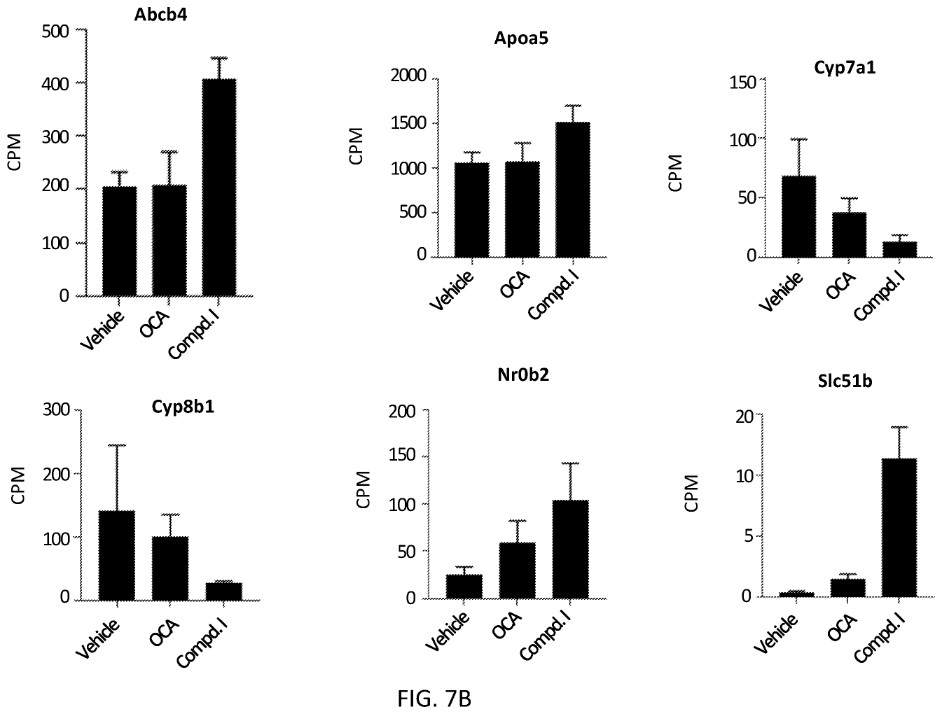

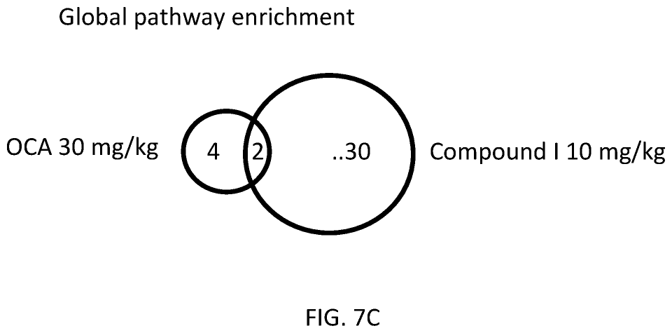

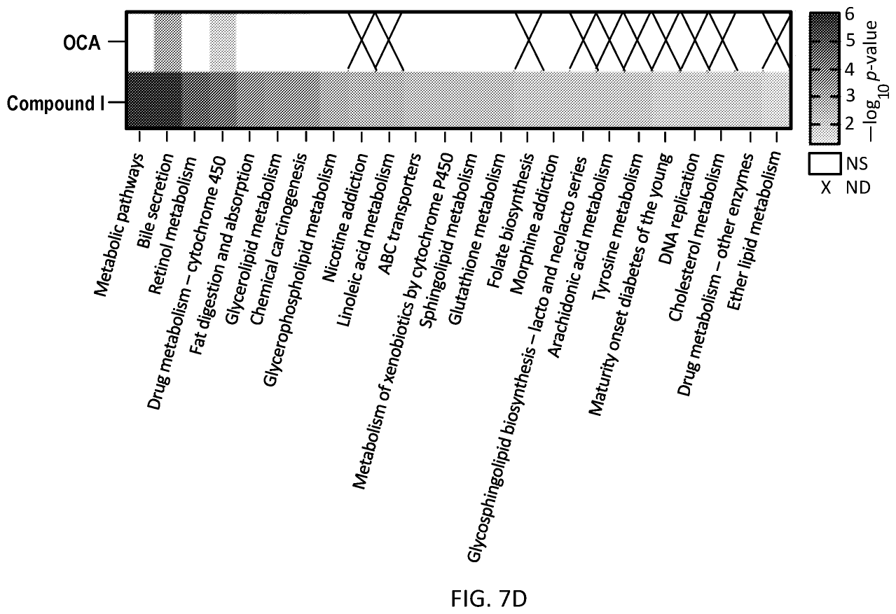

For RNAseq analysis, mRNA was extracted from total liver and sequenced using standard Illumina library preparation and sequencing protocols. Differentially expressed genes (DEG) were determined using RSEM and edgeR software packages and analyzed using Advaita Bio's iPathwayGuide software. Results are shown in FIG. 7A-7D, which indicate that Compound I modulates a significantly higher number of genes and metabolic pathways relevant to NASH compared to OCA. FIG. 7A shows that administration of Compound I modulates expression of 500 NASH-related genes, OCA modulates expression of 44 NASH-related genes, including 37 common NAS-related genes modulated by both Compound I and OCA, relative to vehicle control (fold change≥1.5; q-value<0.05). FIG. 7B shows average expression levels (as shown by CPM value) of select FXR-related genes in vehicle, OCA, and Compound I treated mice. FIG. 7C shows that administration of Compound I causes enrichment of 32 global pathways and that administration of OCA causes enrichment of 6 global pathways, including 2 common global pathways to both Compound I and OCA administration. FIG. 7D shows the 25 pathways most statistically enriched upon Compound I administration, and compares the enrichment of those pathways to the enrichment upon OCA administration. Overall, RNAseq analysis of livers from mice treated with Compound I showed a more robust modulation of FXR-related genes and metabolic pathways relevant to non-alcoholic fatty liver disease compared to OCA treatment.

### Example 9: Clinical Study

First Study. Heathy human volunteer subjects were orally dosed on a daily basis with Compound I at 5 mg (n=9), 75 mg (n=9), 200 mg, or 400 mg (n=18), or received a placebo (n=12) for 14 days. During this study, no incidences of pruritus were observed.

Second Study. Compound I was administered daily for 7 days at oral doses of 25 mg (n=11), 75 mg (n=10), or 150 mg (n=10), or received a placebo (n=5) to human subjects. 7-alpha-hydroxy-4-cholesten-3-one (7α-C4) levels in the patients were periodically measured, as shown in Table 5, which indicated that levels were suppressed by Compound I. In a separate study published by an independent group, FXR agonist MET409 (Metacrine) was reportedly administered daily to healthy human volunteers at doses of 20 mg 40 mg, 50 mg, 80 mg, 100 mg, or 150 mg, and 7α-C4 levels measured as shown in Table 5. See Chen et al., MET409, an Optimized Sustained FXR Agonist, Was Safe and Well-Tolerated in a 14-Day Phase 1 Study in Healthy Subjects, The International Liver Congress, Vienna, Austria, Apr. 10-14, 2019. While pruritus was observed in subjects receiving MET409 at doses of 100 mg or greater, no pruritus was observed for subjects taking the highest doses of Compound I. Other FXR agonists, such as cilofexor, tropifexor, OCA, ED-305 (Enanta) are all known to result in pruritus in longer term studies.

TABLE 5
Comparison of MET409 and Compound I

| | MET409 | | | Compound I | | |
|---|---|---|---|---|---|---|
| | 50 mg | 80 mg | 100 mg | 25 | 75 | 150 |
| Parameters | MET409 | MET409 | MET409 | mg | mg | mg |
| AUC | 6404 | 12479 | 16519 | 645 | 1480 | 2164 |
| ng*h/ml | | | | | | |
| %7α-C4 | 85% | 96% | 99% | 75% | 82% | 93% |
| suppression | | | | | | |
| at nadir | | | | | | |
| AUC/%7α- | 75 | 130 | 166 | 8.6 | 18 | 23 |
| C4 ratio | | | | | | |
| Pruritus | No | No | Yes | No | No | No |

### Example 10: Mouse Model of NASH

The effect of Compound I on NASH was assessed using a mouse model, in which NASH is induced by a high fat diet in combination with CCl₄ administration.

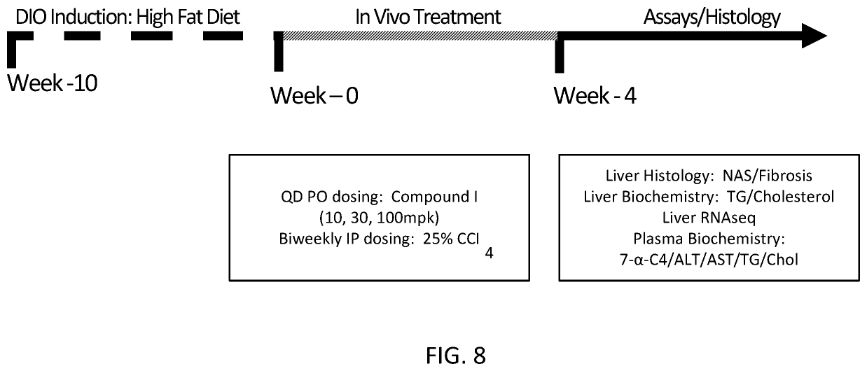

Mice C57/BL6J mice were fed a high fat diet (D12492, Research Diet, fat/protein/carbohydrate 60/20/20 Kcal %, 10 w) to induce obesity (>36 g mouse) prior to daily oral Compound I and biweekly intraperitoneal carbon tetrachloride (CCl₄) treatment for four weeks. FIG. 8. Compound I was administered at a dose of 10, 30, and 100 mg/kg.

Following 28 days of Compound I dosing, serum lipids, serum transaminases and liver lipids were analyzed. Hematoxylin & Eosin (H&E) and Sirius Red histological staining of liver tissue was used to quantitate NAFLD activity score (NAS), steatosis, ballooning, inflammation and fibrosis. Plasma 7-alpha-hydroxy-4-cholesten-3-one (7α-C4) was measured as a biomarker of FXR activation. Gene expression of RNA was analyzed by RT-qPCR and RNAseq.

Nonalcoholic Fatty Liver Disease Activity Score (NAS) is a composite score used to assess NASH. NAS is calculated based upon liver steatosis, inflammation, and ballooning and was determined by analysis of liver tissue histology using H&E stain. Specifically, inflammation score was calculated based upon H&E staining: Score 0, none; 1, <2 foci per 200X field; 2, 2-4 foci per 200X field; 3, >4 foci per 200X field. Steatosis score was calculated by H&E staining as follows: Score 0,<5%; 1,5-33%; 2, >33-66%; 3, >66%). Hepatocellular ballooning is a form of liver cell injury associated with cell swelling and is also measured by H&E stained liver sections. The ballooning score is calculated as follows: 0-no hepatocyte ballooning; 1-few ballooning hepatocytes; 2-many hepatocytes with prominent ballooning.

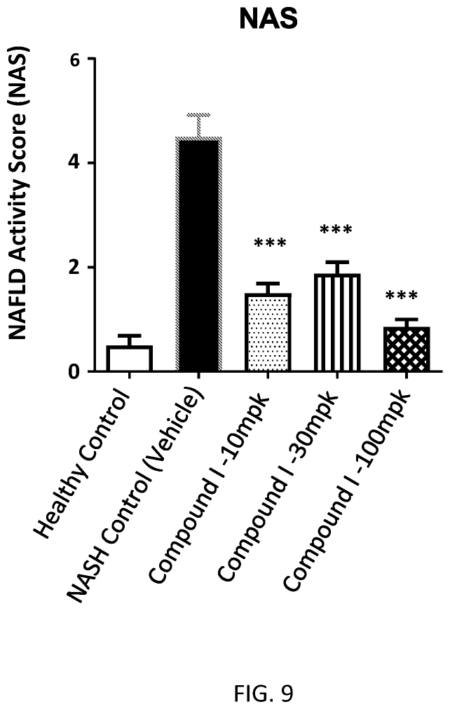

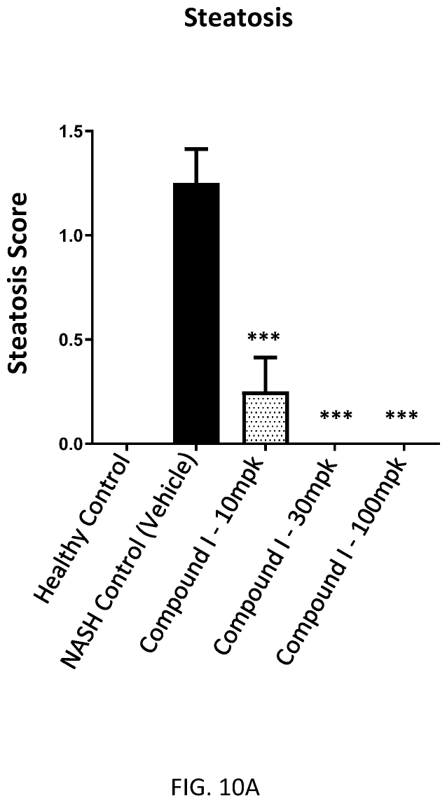

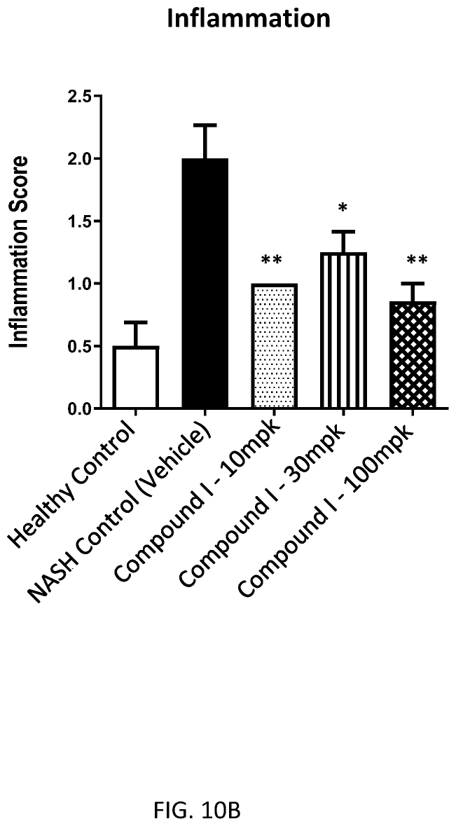

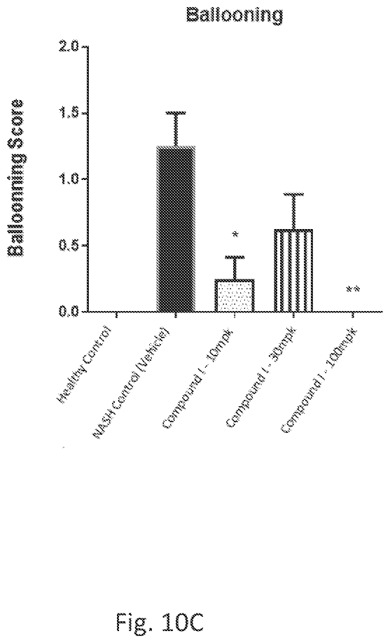

As shown in FIG. 9, mice treated with 10, 30, or 100 mg/kg Compound I had a significantly lower NAS score as compared to untreated NASH mice. Treatment with Compound I also significantly reduced steatosis, inflammation and ballooning compared to untreated NASH mice. FIG. 10A-C.

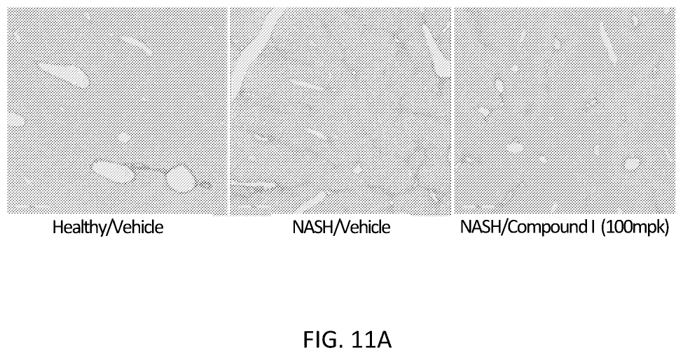

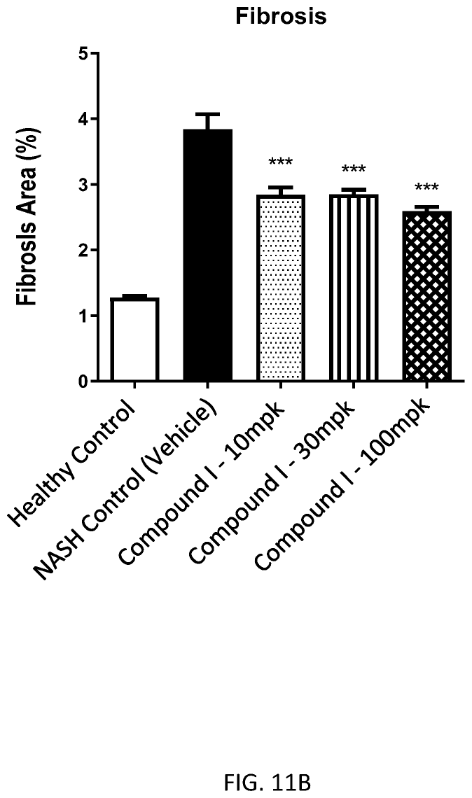

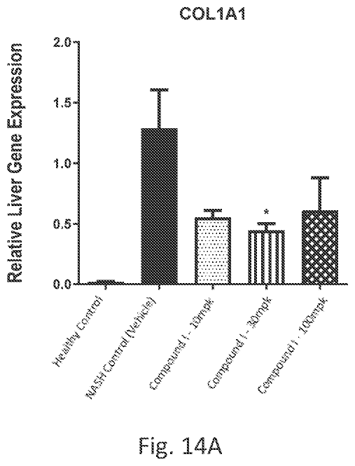

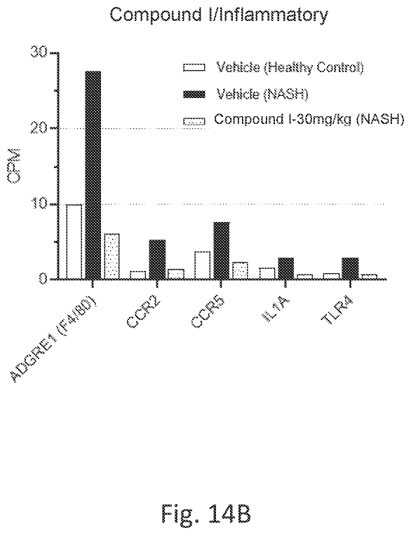

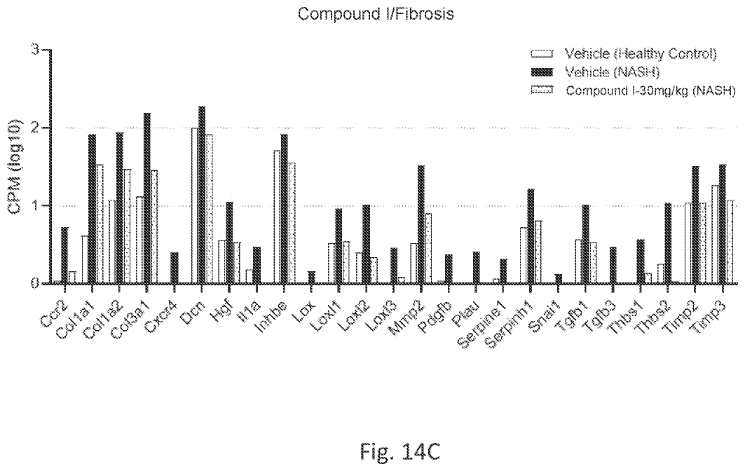

Liver fibrosis was quantified by histological analysis of the percentage of Sirius Red-positive liver sections. FIG. 11A shows representative histology for healthy mice, NASH mice, and NASH mice treated with Compound I at 100 mg/kg. FIG. 11B shows quantification of the fibrosis area of mice treated with Compound I. Treatment with 10, 30 or 100 mg/kg Compound I resulted in statistically significant reduced fibrosis compared to untreated NASH control. As shown in FIG. 14A, Compound I administered at 10, 30, or 100 mg/kg resulted in decreased collagen, type 1, alpha 1 expression in the liver as compared to control NASH mice.

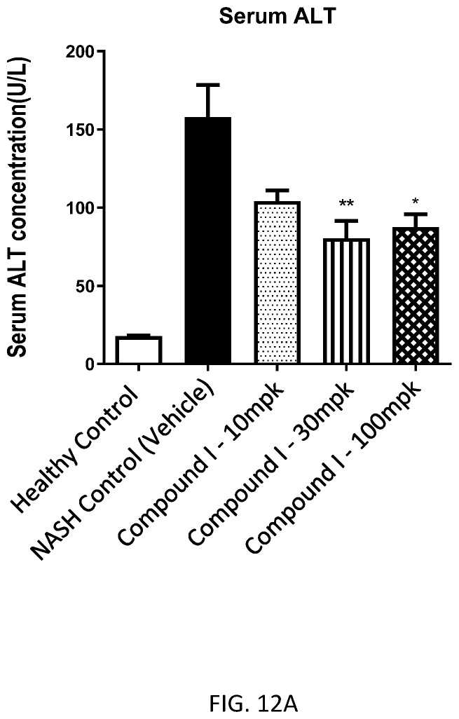

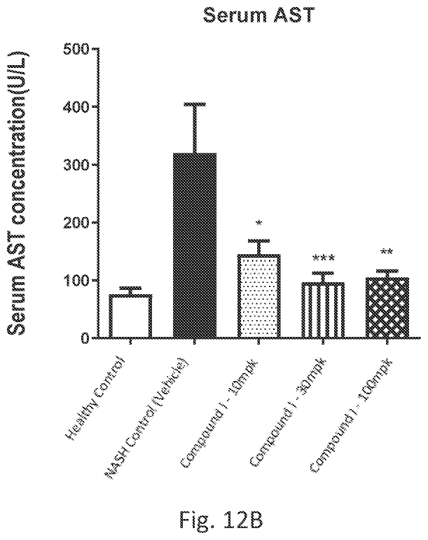

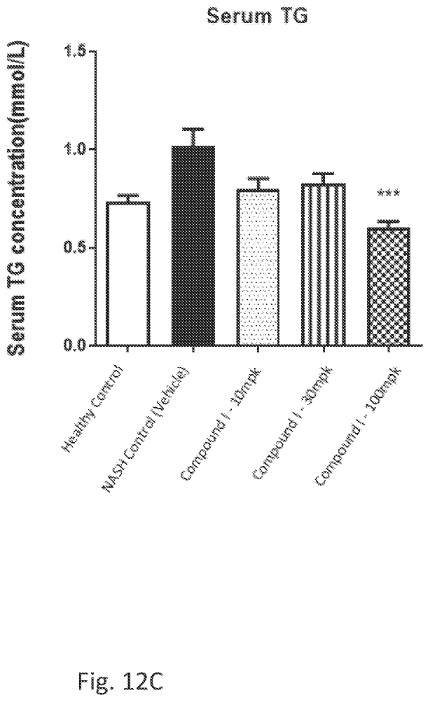

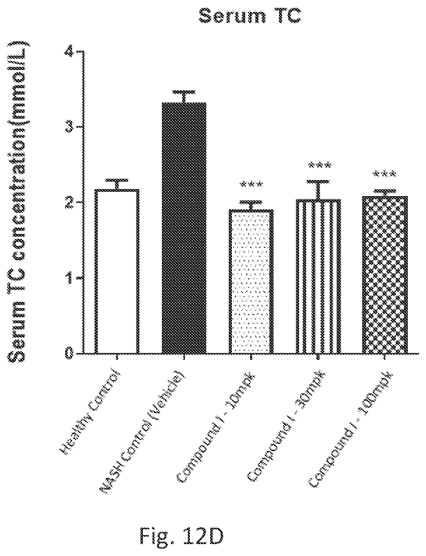

After treatment, serum was analyzed for alanine amino transferase (ALT), aspartate amino transferase (AST), triglyceride, and total cholesterol levels. As shown in FIG. 12A and FIG. 12B serum ALT and AST levels were reduced in mice treated with Compound I. FIG. 12C shows a statically significant reduction in serum triglyceride concentration in mice treated with 100 mg/kg Compound I. FIG. 12D shows statistically significant reduction of total cholesterol level in mice treated with 10, 30, and 100 mg/kg Compound I.

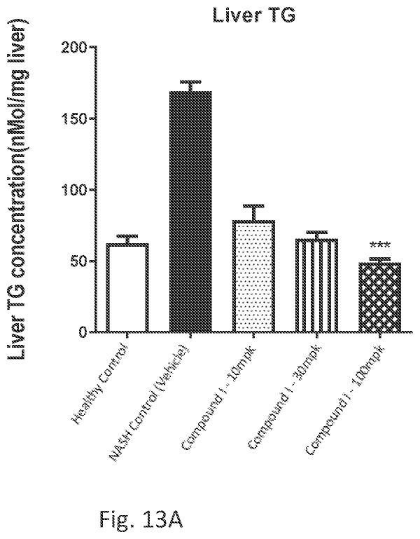

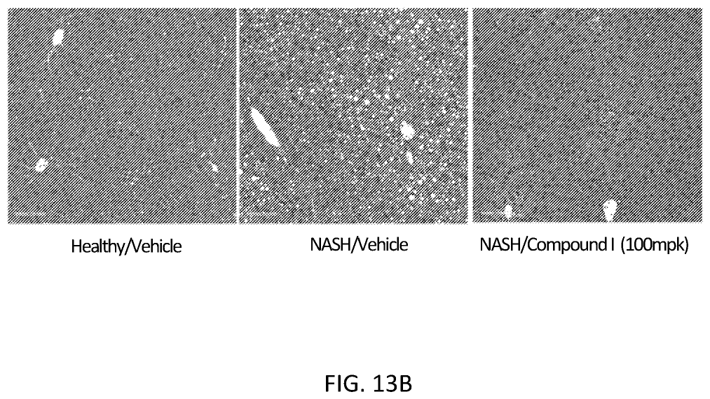

Liver triglycerides were measured from liver tissue using a biochemical analyzer (Hitachi-700). FIG. 13A shows the concentration of liver triglycerides in control mice or mice treated with 10, 30, or 100 mg/kg Compound I. Mice treated with 100 mg/kg Compound I showed statistically significant reduced triglyceride levels. FIG. 13B shows a representative histology section.

The effect of Compound I on gene expression was analyzed using RT-qPCR or RNA-seq of liver samples (FIG. 14A-C and Table 6). Table 6 shows the effect of Compound Ion FXR-regulated gene expression in the liver. The expression level of each indicated gene (as defined by gene count per million (CPM) value) after treatment with compound I was divided by the expression level of that gene in vehicle treated animals to determine the activity of Compound I relative to vehicle.

TABLE 6
Expression of FXR-target, inflammatory, and fibrosis genes

| Gene | Compound I (30 mg/kg) Relative to Vehicle |
|---|---|
| SHP | 4.6 |
| BSEP | 5.1 |
| OST-B | 135.7 |
| CYP7A1 | 0.02 |
| CYP8B1 | 0.007 |

EC₅₀ concentration of Compound I for FXR was determined by a fluorescence-based FXR coactivation assay. Half-log serial dilutions of Compound I or OCA (obeticholic acid, a known FXR agonist) (10 μM-3 nM) were incubated with human FXR ligand binding domain produced in Sf9 insect cells, labeled coactivator SRC-1 peptide and TR-FRET Coregulator Buffer G for 1 h at 25° C. TGR5 activity was measured using a cell-based cAMP assay. See Kawamata et al JBC 278 (11)935-440 (2003). Half-log serial dilutions of Compound I or OCA (10 μM-3 nM) were added to Chinese Hamster Ovary cells expressing recombinant human TGR5. After 30 min at RT, cAMP was measured using an HTRF readout. EC₅₀ values for FXR-regulated gene expression were determined using a cell-based RNA assay. Half-log serial dilutions of Compound I or OCA (3 μM-3 nM) were added to human HuH7 hepatoma cells. After 11 h at 37° C., RNA was isolated and analyzed by RT-qPCR using primers to FXR-related genes: small heterodimer partner (SHP), bile salt export pump (BSEP) and fibroblast growth factor 19 (FGF-19).

As shown in Table 7, Compound I is a potent and selective FXR agonist.

TABLE 7
EC₅₀ of Compound I

| | EC₅₀ of | |
|---|---|---|
| | Compound I | OCA EC₅₀ |
| Assay | (nM) | (nM) |
| FXR Agonist | 57 | 73 |
| TGR5 Agonist | >10,000 | 770 |
| SHP Gene induction/HuH7 | 50 | 200 |
| BSEP Gene induction/HuH7 | 40 | 200 |
| FGF-19 Gene | 40 | 130 |
| Induction/HuH7 | | |

In summary, Compound I is a potent and selective FXR agonist. Compound I reduced expression of inflammatory and fibrosis related genes and strongly suppressed liver steatosis, inflammation, ballooning, and fibrosis in a mouse model of NASH.

### Example 11: Pharmacokinetic Study

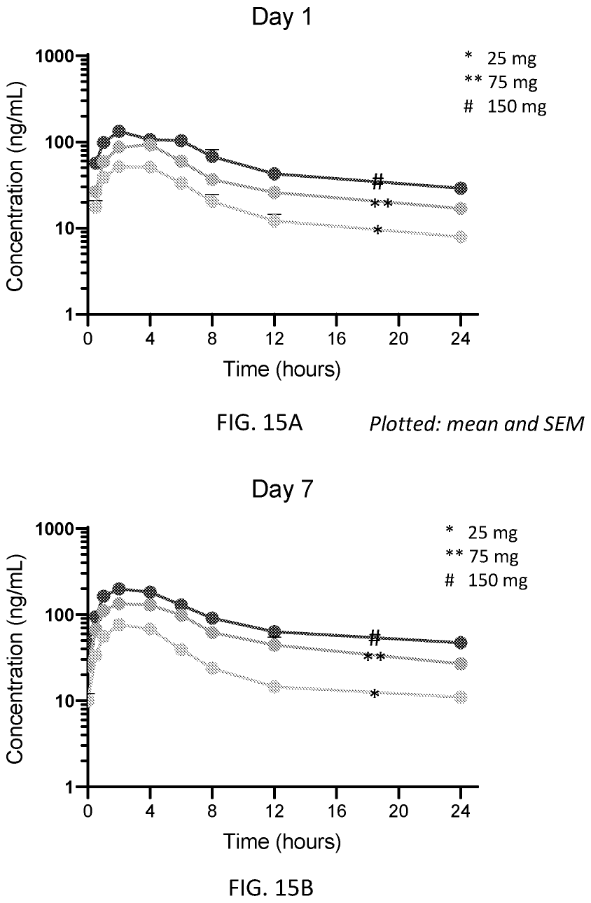

36 healthy subjects were randomized to 4 dosing groups, each of which received placebo, 25 mg, 75 mg, or 150 mg Compound I QD for 7 days. Serum levels of Compound I in the subjects were periodically measured. Mean serum concentrations of Compound I on Day 1 and Day 7 are shown in FIGS. 15A and 15B, respectively.

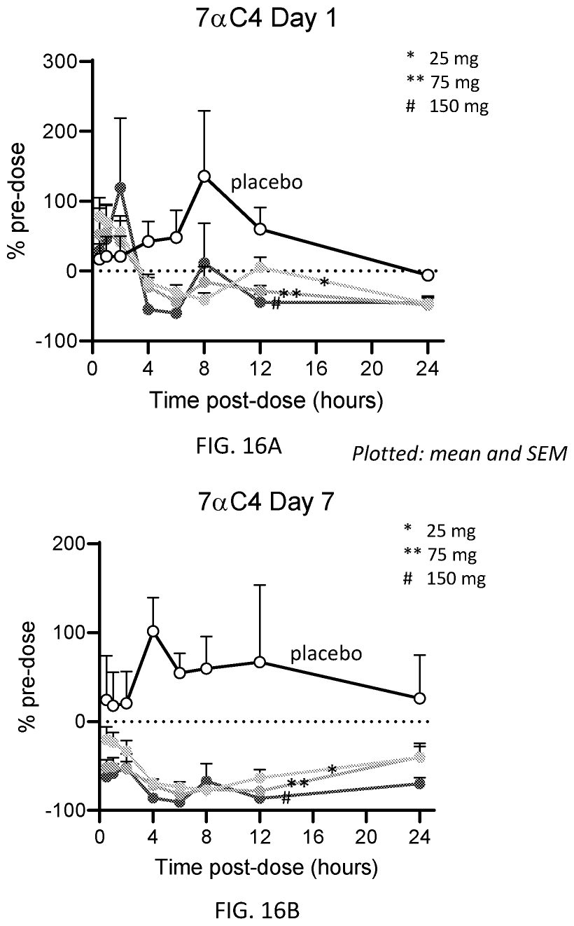

Serum levels of 7-alpha-hydroxy-4-cholesten-3-one (7α-C4) in the subjects were periodically measured. FIGS. 16A and 16B show changes of mean serum levels of 7α-C4 from the pre-dose baseline in different dosing groups on Day 1 and Day 7, respectively. Subjects in the placebo group experienced a post prandial 7α-C4 spike which was inhibited in Compound I dosed subjects. After a single dose on Day 1, a partial suppression of serum 7α-C4 levels was observed in all Compound I dosed groups. On Day 7, maximum suppression of serum 7α-C4 levels was observed six hours after dosing corresponding to a reduction of 74%, 82%, and 91% in the 25, 75, and 150 mg dosing groups, respectively. During each 24-hour dosing period, the mean serum levels of Compound I in all Compound I dosed groups generally did not exceed the EC₅₀ of Compound I. Without being bound by the theory, the reduction of 7α-C4 is believed to be associated with higher concentrations of Compound I in liver due to its preferential distribution in liver.

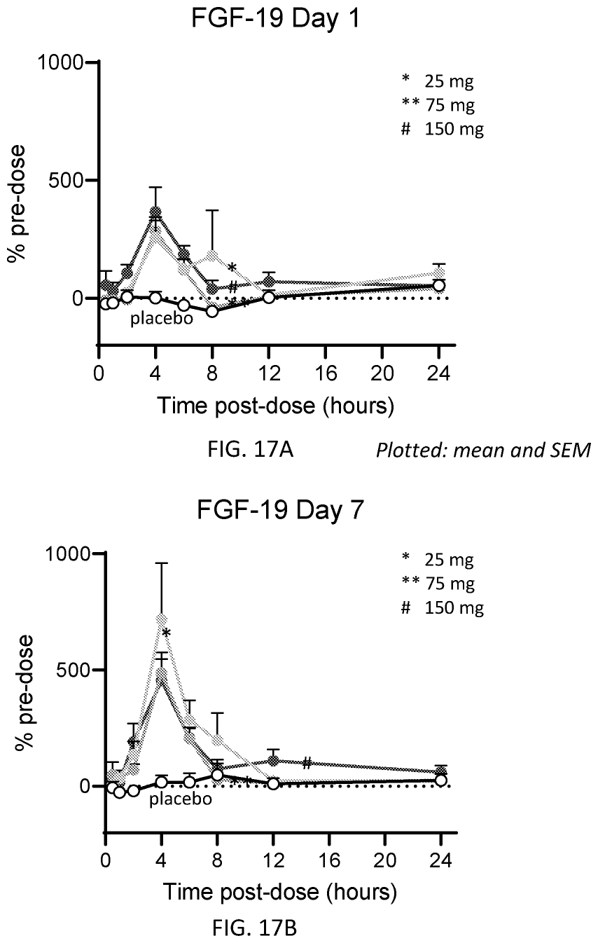

Serum levels of FGF-19 in the subjects were periodically measured. FIGS. 17A and 17B show changes of mean serum levels of FGF-19 from the pre-dose baseline in different dosing groups on Day 1 and Day 7, respectively. Maximum increases from baseline of 718%, 486%, and 454% in serum FGF 19 levels were observed at four hours after dosing on Day 7.

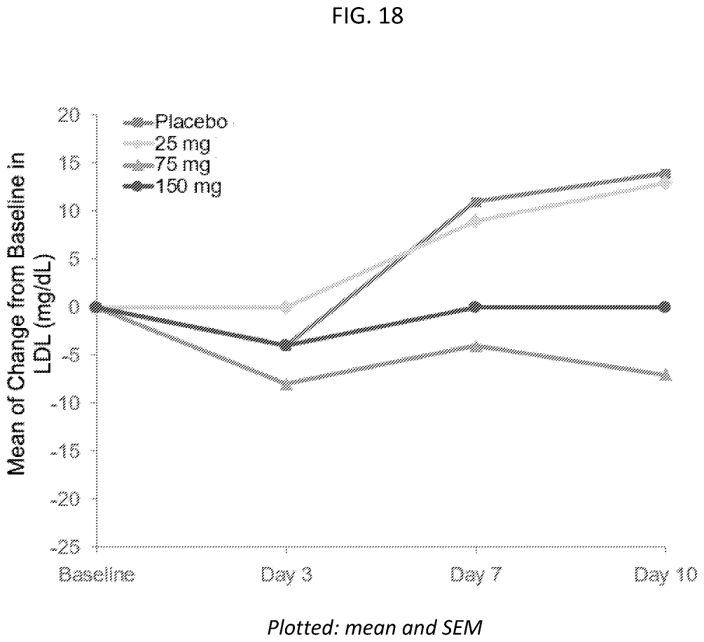

Serum levels of low-density lipoprotein (LDL) in the subjects were periodically measured. FIG. 18 shows changes of mean serum levels of low-density lipoprotein (LDL) from the pre-dose baseline in different dosing groups. Serum LDL changes above baseline were minimal in Compound I dosed groups and did not exceed the LDL elevations observed in subjects dosed with placebo.

Safety was assessed during dosing and for 10 (±1) days after dosing. Compound I was safe and well tolerated in healthy volunteers at all doses with no reports of pruritus.

All references throughout, such as publications, patents, patent applications and published patent applications, are incorporated herein by reference in their entireties.

What is claimed is:

1. A method of treating a liver disorder in a patient in need thereof comprising administering a therapeutically effective amount of a compound of Formula (I) 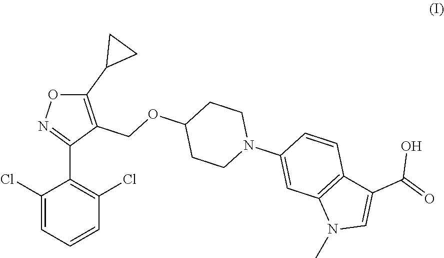 [SMILES: Cn1cc(C(=O)O)c2ccc(N3CCC(OCc4c(-c5c(Cl)cccc5Cl)noc4C4CC4)CC3)cc21]
or a pharmaceutically acceptable salt thereof, wherein the liver disorder is selected from liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH).

2. A method of treating a liver disorder in a patient in need thereof with a Farnesoid X Receptor (FXR) agonist, comprising administering a therapeutically effective amount of the FXR agonist, wherein the FXR agonist is a compound of Formula (I) 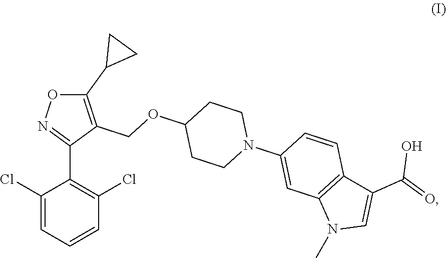 [SMILES: Cn1cc(C(=O)O)c2ccc(N3CCC(OCc4c(-c5c(Cl)cccc5Cl)noc4C4CC4)CC3)cc21]
or a pharmaceutically acceptable salt thereof, and wherein the patient has discontinued one or more prior therapies with another FXR agonist other than a compound of Formula (I) or a pharmaceutically acceptable salt thereof, wherein the liver disorder is selected from liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH).

3. The method of claim 2, wherein the patient suffered from pruritus during the one or more prior therapies.

4. A method of impeding or slowing the progression of non-alcoholic fatty liver disease (NAFLD) to non-alcoholic steatohepatitis (NASH) in a patient in need thereof comprising administering a therapeutically effective amount of a compound of Formula (I) 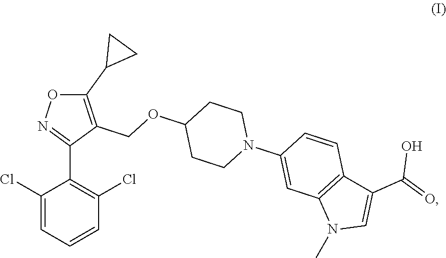 [SMILES: Cn1cc(C(=O)O)c2ccc(N3CCC(OCc4c(-c5c(Cl)cccc5Cl)noc4C4CC4)CC3)cc21]
or a pharmaceutically acceptable salt thereof.

5. A method of impeding or slowing the progression of NASH in a patient in need thereof comprising administering a therapeutically effective amount of a compound of Formula (I) 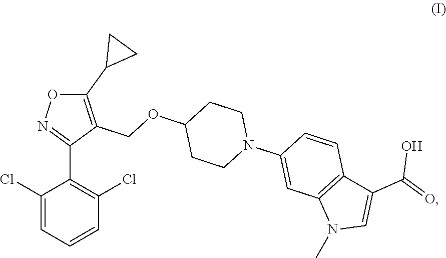 [SMILES: Cn1cc(C(=O)O)c2ccc(N3CCC(OCc4c(-c5c(Cl)cccc5Cl)noc4C4CC4)CC3)cc21]
or a pharmaceutically acceptable salt thereof.

6. A method of treating a liver disorder in a patient in need thereof with a Farnesoid X Receptor (FXR) agonist that preferentially concentrates in liver tissue over one or more of kidney, lung, heart, and skin tissues, the method comprising administering a therapeutically effective amount of the FXR agonist, wherein the FXR agonist is a compound of Formula (I) 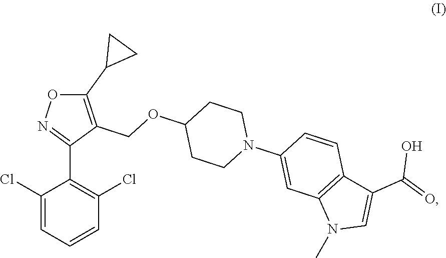 [SMILES: Cn1cc(C(=O)O)c2ccc(N3CCC(OCc4c(-c5c(Cl)cccc5Cl)noc4C4CC4)CC3)cc21]
or a pharmaceutically acceptable salt thereof, wherein the liver disorder is selected from liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH).

7. The method of any one of claims 1-6, wherein the administration does not result in pruritus in the patient greater than Grade 2 in severity.

8. The method of any one of claims 1-7, wherein the administration does not result in pruritus in the patient greater than Grade 1 in severity.

9. The method of any one of claims 1-8, wherein the administration does not result in pruritus in the patient.

10. A method of treating a liver disorder with an FXR agonist that does not result in detectable pruritus in a patient in need thereof, the method comprising administering a therapeutically effective amount of the FXR agonist, wherein the FXR agonist is a compound of Formula (I) 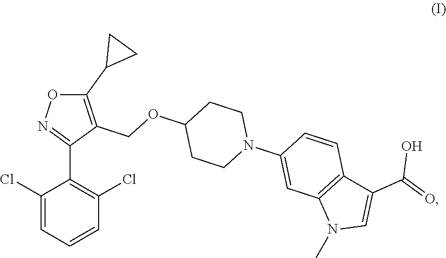 [SMILES: Cn1cc(C(=O)O)c2ccc(N3CCC(OCc4c(-c5c(Cl)cccc5Cl)noc4C4CC4)CC3)cc21]
or a pharmaceutically acceptable salt thereof, wherein the liver disorder is selected from liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH).

11. The method of any one of claim 1-3, or 6-10, wherein the liver disorder is NAFLD.

12. The method of any one of claim 1-3, or 6-10, wherein the liver disorder is NASH.

13. The method of any one of claim 1-3, or 6-10, wherein the liver disorder is PSC.

14. The method of any one of claim 1-3, or 6-10, wherein the liver disorder is PBC.

15. The method of any one of claims 1-14, wherein the administration results in a liver concentration to plasma concentration ratio of the compound of Formula (I) of 10 or greater.

16. The method of any one of claims 1-15, wherein the therapeutically effective amount is 0.5 μg/day-600 mg/day.

17. The method of any one of claims 1-16, wherein the therapeutically effective amount is 0.5 μg/day-20 mg/day.

18. The method of any one of claims 1-17, wherein the therapeutically effective amount is 0.5 μg/day-4 mg/day.

19. The method of any one of claims 1-18, wherein the administration comprises administering the compound of Formula (I), or a pharmaceutically acceptable salt thereof, daily for a treatment period of one or more weeks.

20. The method of claim 19, wherein the compound of Formula (I), or a pharmaceutically acceptable salt thereof, is administered once daily or twice daily.

21. The method of claim 19 or 20, wherein the amount of the compound of Formula (I), or a pharmaceutically acceptable salt thereof, administered on day 1 of the treatment period is greater than or equal to the amount administered on all subsequent days of the treatment period.

22. The method of any one of claims 19-21, wherein the amount of the compound of Formula (I), or a pharmaceutically acceptable salt thereof, administered on day 1 of the treatment period is equal to the amount administered on all subsequent days of the treatment period.

23. The method of any one of claims 1-22, wherein the treatment period is one or more months.

24. The method of any one of claims 1-23, wherein the treatment period is the remaining lifespan of the patient.

25. The method of any one of claims 1-24, wherein the patient is obese.

26. The method of any one of claims 1-24, wherein the patient is not obese.

27. The method of any one of claims 1-26, wherein the patient also has diabetes mellitus and/or a cardiovascular disorder.

28. The method of any one of claims 1-27, wherein the patient is at risk for developing an adverse effect affecting one or more of the kidney, lung, heart, and skin.

29. The method of any one of claims 1-28, wherein the patient is 2-17 years old.

30. The method of any one of claims 1-28, wherein the patient is 18-54 years old.

31. The method of any one of claims 1-28, wherein the patient is 65 or more years old.

32. The method of any one of claims 1-31, wherein the patient has had a liver transplant.

33. The method of any one of claims 1-32, wherein the patient's alkaline phosphatase, gamma-glutamyl transferase (GGT), alanine aminotransferase (ALT) and/or aspartate aminotransferase (AST) levels are elevated.

34. The method of any one of claims 1-33, wherein the method does not comprise administering an antihistamine, an immunosuppressant, a steroid, rifampicin, an opioid antagonists, or a selective serotonin reuptake inhibitor (SSRI).

35. The method of any one of claims 1-34, wherein the NAS score of the patient is decreased.

36. The method of any one of claims 1-35, wherein TGR5 signaling is not activated.

37. The method of any one of claims 1-36, wherein the expression level of a marker of fibrosis is decreased.

38. The method of any one of claims 1-37, wherein the expression level of Ccr2, Col1a1, Col1a2, Col1a3, Cxcr3, Dcn, Hgf, Il1a, Inhbe, Lox, Loxl1, Loxl2, Loxl3, Mmp2, Pdgfb, Plau, Serpine1, Perpinh1, Snai, Tgfb1, Tgfb3, Thbs1, Thbs2, Timp2, and/or Timp3 is reduced.

39. The method of any one of claims 1-38, wherein the expression level of a marker of liver inflammation is decreased.

40. The method of any one of claims 1-39, wherein the level of Adgre1, Ccr2, Ccr5, Il1A, and/or Tlr4 is reduced.
